(19) United States
(12) Patent Application Publication
(10) Pub. No.: US 2023/0380964 A1
(43) Pub. Date: Nov. 30, 2023

(54) PROSTHETIC VALVES

(71) Applicant: EDWARDS LIFESCIENCES CORPORATION, Irvine, CA (US)

(72) Inventors: Michael G. Valdez, Riverside, CA (US); Tiana Tran, Westminster, CA (US)

(21) Appl. No.: 18/448,726

(22) Filed: Aug. 11, 2023

Related U.S. Application Data
(63) Continuation of application No. PCT/US2022/014977, filed on Feb. 2, 2022.
(60) Provisional application No. 63/148,228, filed on Feb. 11, 2021.

(51) Int. Cl. A61F 2/24

(52) U.S. Cl. CPC A61F 2/2418; A61F 2/246; A61F 2210/0014

(57) ABSTRACT

A prosthetic valve can comprise a body member having a first end and a second end, the body member defining a conduit and being configured to assume a compressed configuration and an expanded configuration, a lateral dimension of the body member being larger in the expanded configuration than that in the compressed configuration. An elongate member can have a first end associated with the first end of the body member, the elongate member being configured to move between a longitudinal orientation and a diametrical orientation. A covering can be coupled to the elongate member and configured to cover at least a portion of the conduit at the first end of the body member when the elongate member is in the diametrical orientation.

### RELATED APPLICATIONS

This application is a continuation of International Patent Application No. PCT/US2022/014977, filed Feb. 2, 2022, and entitled PROSTHETIC VALVES, which claims priority based on U.S. Provisional Patent Application Ser. No. 63/148,228, filed Feb. 11, 2021, and entitled PROSTHETIC VALVES, the complete disclosures of each of which are hereby incorporated by reference herein in their entirety.

### BACKGROUND

### Field

The present disclosure generally relates to the field of medical prosthetic valves.

### Description of Related Art

Prosthetic valves can be positioned at various sites in the human body to regulate fluid flow. Prosthetic valves can be used in the heart to provide control of the flow of blood. Artificial control of blood flow with prosthetic valves within the heart can be used to treat any number of conditions, including elevated blood pressure.

### SUMMARY

Described herein are devices, systems, and methods relating to prosthetic valves configured to be able to change from a compressed configuration to an expanded configuration.

In some implementations, a prosthetic valve can comprise a body member comprising a first end and a second end, the body member defining a conduit and being configured to assume a compressed configuration and an expanded configuration, a lateral dimension of the body member being larger in the expanded configuration than that in the compressed configuration. The prosthetic valve can include an elongate member having a first end associated with the first end of the body member, the elongate member being configured to move between a longitudinal orientation and a diametrical orientation; and a covering coupled to the elongate member and configured to cover at least a portion of the conduit at the first end of the body member when the elongate member is in the diametrical orientation.

In some embodiments, when the prosthetic valve is in the expanded configuration, the prosthetic valve is configured to alternate between an open state and a closed state. When the prosthetic valve is in the open state, at least a portion of the covering can be configured to be spaced from the first end of the body member to open the first end to fluid flow, and when the prosthetic valve is in the closed state, the covering can be configured to close off the first end of the body member from fluid flow.

In some embodiments, when the prosthetic valve is in the expanded configuration, the body member forms a cylindrical shape and the elongate member is configured to be positioned across a diameter of the first end of the body member, and the first and second ends of the elongate member are configured to be coupled to diametrically opposing positions on the first end of the body member.

In some embodiments, the elongate member is rotatably coupled to the first end of the body member. In some embodiments, the prosthetic valve comprises a hinge connector rotatably coupling the first end of the elongate member and the first end of the body member. In some embodiments, the prosthetic valve can further comprise a rotation limiter extending from the first end of the body member and configured to contact at least one of the elongate member and the hinge connector to limit rotation of the elongate member relative to the body member.

In some embodiments, the elongate member is integral with the body member and the elongate member and the body member comprise a shape memory material shape set to position the elongate member across the first end of the body member when the prosthetic valve is in the expanded configuration.

In some embodiments, when the prosthetic valve is in the expanded configuration, a second end of the elongate member is coupled to the first end of the body member.

In some embodiments, the covering is in a rolled configuration while the prosthetic valve is in the compressed configuration, the rolled configuration comprising a longitudinal axis parallel to that of the body member. In some embodiments, the covering is mechanically reinforced around at least a portion of an edge portion of the covering.

In some embodiments, the covering is one leaflet, the one leaflet being coupled to the elongate member along a longitudinal dimension of the elongate member, and when the prosthetic valve is in the expanded configuration and the closed state, the one leaflet is configured to be positioned over the first end of the body member to close off the fluid flow.

In some embodiments, the covering comprises a first leaflet and a second leaflet, each of the first and second leaflets being coupled to the elongate member along a longitudinal dimension of the elongate member. When the prosthetic valve is in the expanded configuration and the closed state, the first and second leaflets can each be configured to cover a corresponding portion of the first end of the body member to close off the fluid flow.

In some embodiments, the first and second leaflets are each rotatably coupled to the elongate member. When the prosthetic valve is in the open state, the first and second leaflets can be configured to be pivotally rotated around the elongate member and away from the first end of the body member.

In some embodiments, the first end of the body member comprises an atraumatic configuration.

In some embodiments, the prosthetic valve can further comprise a collar coupled to the first end of the body member, the collar comprising at least a portion extending laterally from an outer edge of the first end of the body member to contact tissue at the target site, wherein the covering is configured to contact the collar while the prosthetic valve is in the closed state. In some embodiments, the collar extends laterally from and around the first end of the body member. In some embodiments, the collar comprises a radiopaque material.

In some embodiments, the prosthetic valve can further comprise a sheath associated with an outer surface of the body member configured to be oriented toward tissue at the target site.

In some embodiments, the body member and covering each comprise shape memory material.

In some implementations, a prosthetic valve can comprise a body member comprising a first end and a second end, the body member defining a conduit extending therethrough and being configured to assume a compressed configuration and an expanded configuration, a lateral dimension of the body member being larger in the expanded configuration than that in the compressed configuration. The prosthetic valve can include a first elongate member comprising a first end and a second end, the first end of the first elongate member being associated with the first end of the body member at a first position on the first end of the body member. A second elongate member can comprise a first end and a second end, the first end of the second elongate member being associated with the first end of the body member at a second position on the first end of the body member, the first and second elongate members being configured to move between a longitudinal orientation and a diametrical orientation. A first covering can be coupled to the first elongate member and a second covering can be coupled to the second elongate member, the first and second coverings being configured to at least partially cover respective portions of the conduit at the first end of the body member when the first and second elongate members are in the diametrical orientation.

In some embodiments, when the prosthetic valve is in the expanded configuration, the prosthetic valve is configured to alternate between an open state and a closed state. When the prosthetic valve is in the open state, at least a portion of the first and second coverings can be configured to be spaced from the first end of the body member to open the first end to fluid flow, and when the prosthetic valve is in the closed state, the first and second coverings can be configured to be positioned over respective portions of the first end of the body member to close off the first end fluid flow.

In some embodiments, when the prosthetic valve is in the expanded configuration, the second ends of the first and second elongate members are coupled to the first end of the body member.

In some embodiments, when the prosthetic valve is in the expanded configuration, the first and second elongate members are positioned across respective portions of the first end of the body member.

In some embodiments, when the prosthetic valve is in the expanded configuration, the first and second elongate members comprise a stacked configuration and are positioned across a same portion of the first end of the body member. In some embodiments, the first end and second end of the first elongate member are coupled to opposing positions on the first end of the body member, and the first end and the second end of the second elongate member are coupled to opposing positions on the first end of the body member. In some embodiments, when the prosthetic valve is in the expanded configuration, the body member forms a cylindrical shape, and the first and second elongate members are configured to be positioned across a diameter of the body member.

In some embodiments, the first and second coverings each comprise a rolled configuration while the prosthetic valve is in the compressed configuration, each rolled configuration comprising a longitudinal axis parallel to that of the body member.

In some embodiments, the first covering comprises a first leaflet and the second covering comprises a second leaflet, the first leaflet being coupled to the first elongate member along a longitudinal dimension of the first elongate member and the second leaflet being coupled to the second elongate member along a longitudinal dimension of the second elongate member. When the prosthetic valve is in the expanded configuration and the closed state, the first and second leaflets can each be configured to cover a corresponding portion of the first end of the body member to close off fluid flow through the first end. In some embodiments, the first and second coverings are each rotatably coupled to the respective elongate member, and when the prosthetic valve is in the open state, the first and second coverings are configured to be pivotally rotated around the respective elongate member and away from the first end of the body member.

In some implementations, a method of delivering a prosthetic valve can comprise positioning a distal portion of a delivery catheter at a target position within a vessel, the prosthetic valve being in a compressed configuration and being within the distal portion of the delivery catheter, and the prosthetic valve comprising an elongate member in a longitudinal orientation and coupled to a first end of a body member. The method can include causing the prosthetic valve to assume an expanded configuration after releasing the prosthetic valve from the delivery catheter, and, in the expanded configuration, the body member comprising a lateral dimension larger than that in the compressed configuration and the elongate member assuming a diametrical orientation and positioned over the first end of the body member. The method can include positioning the prosthetic valve in the expanded configuration at a target site.

In some embodiments, releasing the prosthetic valve from the delivery catheter comprises releasing the prosthetic valve comprising the first end of the body member oriented proximally and the second end of the body member oriented distally.

In some embodiments, the method can further comprise advancing the distal portion of the delivery catheter into a right atrium, advancing the distal portion of the delivery catheter through a septum from the right atrium into a left atrium, and positioning the distal portion of the delivery catheter at a target position in the left atrium adjacent to an opening in the left atrium to a pulmonary vein. Releasing the prosthetic valve can comprise positioning at least a portion of the body member within the pulmonary vein. While positioned at the target site, the prosthetic valve can be configured to be in an open state to open blood flow from the pulmonary vein into the left atrium and in a closed state to close off blood flow from the left atrium into the pulmonary vein.

In some embodiments, the method can further comprise advancing the distal portion of the delivery catheter into a right atrium, and positioning the distal portion of the delivery catheter adjacent to a coronary sinus ostium in the right atrium. Releasing the prosthetic valve can comprise positioning at least a portion of the body member within the coronary sinus. While positioned at the target site, the prosthetic valve can be configured to be in an open state to open blood flow from the coronary sinus into the right atrium and in a closed state to close off blood flow from the right atrium into the coronary sinus.

In some embodiments, the method can further comprise advancing the distal portion of the delivery catheter into a right atrium and from the right atrium into a right ventricle through a tricuspid valve, advancing the distal portion of the delivery catheter through a septum from the right ventricle into a left ventricle, and positioning the distal portion of the delivery catheter at a target position in the left ventricle adjacent to a mitral valve. Releasing the prosthetic valve can comprise positioning at least a portion of the body member within the mitral valve. While positioned at the target site, the prosthetic valve can be configured to be in an open state to open blood flow from the left atrium into the left ventricle and in a closed state to close off blood flow from the left ventricle into the left atrium.

In some embodiments, releasing the prosthetic valve from the delivery catheter comprises releasing the prosthetic valve comprising the first end of the body member oriented distally and the second end of the body member oriented proximally.

In some embodiments, the method can further comprise advancing the distal portion of the delivery catheter into a right atrium, and through a tricuspid valve from the right atrium into a right ventricle, advancing the distal portion of the delivery catheter through a septum from the right ventricle into a left ventricle, and positioning the distal portion of the delivery catheter at a target position in the left ventricle adjacent to an opening in the left ventricle to an aorta. Releasing the prosthetic valve can comprise positioning at least a portion of the body member within an aortic valve. While positioned at the target site, the prosthetic valve can be configured to be in an open state to open blood flow from the left ventricle into the aorta and in a closed state to close off blood flow from the aorta into the left ventricle.

In some embodiments, the method can further comprise advancing the distal portion of the delivery catheter into a right atrium, and positioning the distal portion of the delivery catheter adjacent to a tricuspid valve. Releasing the prosthetic valve can comprise positioning at least a portion of the body member within the tricuspid valve. While positioned at the target site, the prosthetic valve can be configured to be in an open state to open blood flow from the right atrium into the right ventricle and in a closed state to close off blood flow from the right ventricle into the right atrium.

In some embodiments, the method can further comprise advancing the distal portion of the delivery catheter into a right atrium, and advancing the distal portion of the delivery catheter from the right atrium into a left atrium through a septum, and positioning the distal portion of the delivery catheter at a target position adjacent to a mitral valve. Releasing the prosthetic valve can comprise positioning at least a portion of the body member within the mitral valve. While positioned at the target site, the prosthetic valve can be configured to be in an open state to open blood flow from the left atrium into the left ventricle and in a closed state to close off blood flow from the left ventricle into the left atrium.

In some embodiments, the method can further comprise advancing the distal portion of the delivery catheter into an inferior vena cava, and positioning the distal portion of the delivery catheter adjacent to an opening of the inferior vena cava into a right atrium. Releasing the prosthetic valve can comprise positioning at least a portion of the body member within the inferior vena cava. While positioned at the target site, the prosthetic valve can be configured to be in an open state to open blood flow from the inferior vena cava into the right atrium and in a closed state to close off blood flow from the right atrium into the inferior vena cava.

In some embodiments, the method can further comprise advancing the distal portion of the delivery catheter into a superior vena cava, and positioning the distal portion of the delivery catheter adjacent to an opening of the superior vena cava into a right atrium. Releasing the prosthetic valve can comprise positioning at least a portion of the body member within the superior vena cava. While positioned at the target site, the prosthetic valve can be configured to be in an open state to open blood flow from the superior vena cava into the right atrium and in a closed state to close off blood flow from the right atrium into the superior vena cava.

In some embodiments, releasing the prosthetic valve comprises translating the prosthetic valve distally relative to the delivery catheter, and translating the prosthetic valve comprises contacting the prosthetic valve with a pusher shaft and pushing the prosthetic valve distally with the pusher shaft.

In some embodiments, positioning the prosthetic valve in the expanded configuration at the target site comprises positioning the prosthetic valve in the heart.

For purposes of summarizing the disclosure, certain aspects, advantages and novel features have been described herein. It is to be understood that not necessarily all such advantages may be achieved in accordance with any particular embodiment. Thus, the disclosed embodiments may be carried out in a manner that achieves or optimizes one advantage or group of advantages as taught herein without necessarily achieving other advantages as may be taught or suggested herein.

### BRIEF DESCRIPTION OF THE DRAWINGS

Various embodiments are depicted in the accompanying drawings for illustrative purposes and should in no way be interpreted as limiting the scope of the inventions. In addition, various features of different disclosed embodiments can be combined to form additional embodiments, which are part of this disclosure. Throughout the drawings, reference numbers may be reused to indicate correspondence between reference elements. However, it should be understood that the use of similar reference numbers in connection with multiple drawings does not necessarily imply similarity between respective embodiments associated therewith. Furthermore, it should be understood that the features of the respective drawings are not necessarily drawn to scale, and the illustrated sizes thereof are presented for the purpose of illustration of inventive aspects thereof. Generally, certain of the illustrated features may be relatively smaller than as illustrated in some embodiments or configurations.

FIG. 1 is a cross-sectional view of a human heart.

FIGS. 2A and 2B are perspective views of an example of a prosthetic valve in the expanded configuration in accordance with one or more embodiments.

FIGS. 3A and 3B are side views of the prosthetic valve of FIG. 2 positioned at a target site.

FIG. 4A is a perspective view of the prosthetic valve of FIG. 2 in a compressed configuration, and FIG. 4B is a perspective view of the prosthetic valve of FIG. 2 in an expanded configuration.

FIGS. 5A, 5B, 5C and 5D show cross-sectional side views of an example of a prosthetic valve delivery system used to position the prosthetic valve of FIG. 2 at a target site, in accordance with one or more embodiments.

FIG. 6 shows examples of various target sites within the heart at which a prosthetic valve can be positioned.

FIG. 7 shows examples of various target sites within the heart at which a prosthetic valve can be positioned.

FIG. 8A is a side view of an example of a prosthetic valve in an expanded configuration in accordance with one or more embodiments. FIG. 8B is a top-down view, and FIG. 8C is a perspective view, of the prosthetic valve shown in FIG. 8A. FIG. 8D is another side view of the prosthetic valve of FIG. 8A.

FIG. 9A is a top-down view, and FIG. 9B is a side view, of the prosthetic valve of FIG. 8 in an expanded configuration and a closed state.

FIG. 10A is a top-down view, and FIG. 10B is a side view, of the prosthetic valve of FIG. 8 in an expanded configuration and an open state.

FIG. 11A is a top-down view, and FIG. 11B is a side view, of the prosthetic valve of FIG. 8 in a compressed configuration.

FIG. 12A is a top-down view, and FIGS. 12B and 12C are side views, of another example of a prosthetic valve.

FIG. 13A is a top-down view, and FIG. 13B is a side view, of the prosthetic valve of FIG. 12 in a closed state.

FIG. 14A is a top-down view, and FIG. 14B is a side view, of the prosthetic valve of FIG. 12 in an open state.

FIG. 15A is a top-down view, and FIGS. 15B and 15C are side views, of the prosthetic valve.

FIG. 16A is a top-down view, and FIG. 16B is a side view, of the prosthetic valve of FIG. 15 in a closed state.

FIG. 17A is a top-down view, and FIG. 17B is a side view, of the prosthetic valve of FIG. 15 in an open state.

FIG. 18 shows an example of a prosthetic valve comprising a hinge connector.

FIG. 19 shows an example of a prosthetic valve comprising a hinge connector and a rotation limiter.

FIG. 20 is a process flow diagram of an example of a process for deploying a prosthetic valve as described herein.

### DETAILED DESCRIPTION

The headings provided herein are for convenience only and do not necessarily affect the scope or meaning of the claimed invention.

The present disclosure relates to devices, systems and methods for providing one or more prosthetic valves configured to be transformable from a compressed configuration to an expanded configuration. In the compressed configuration, a prosthetic valve can be more compact and have a reduced profile, such as compared to that of the expanded configuration. The prosthetic valve can have an elongate configuration while compressed. A compressed prosthetic valve can subsequently expand along one or more width directions to transform into the expanded configuration. The prosthetic valve can be in the compressed configuration while being delivered to a target site and can be in the expanded configuration at the target site.

Although certain preferred embodiments and examples are disclosed below, inventive subject matter extends beyond the specifically disclosed embodiments to other alternative embodiments and/or uses and to modifications and equivalents thereof. Thus, the scope of the claims that may arise herefrom is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components. For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

Certain standard anatomical terms of location are used herein to refer to the anatomy of animals, and namely humans, with respect to the preferred embodiments. Although certain spatially relative terms, such as “outer,” “inner,” “upper,” “lower,” “below,” “above,” “vertical,” “horizontal,” “top,” “bottom,” and similar terms, are used herein to describe a spatial relationship of one device/element or anatomical structure to another device/element or anatomical structure, it is understood that these terms are used herein for ease of description to describe the positional relationship between element(s)/structures(s), as illustrated in the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa.

Prosthetic valves can be positioned at various sites in the human body to regulate fluid flow. For example, prosthetic valves can be used in and/or around the heart to improve control of the flow of blood into, out of and/or within the heart. Improved control in the flow of blood within the heart can be used to treat any number of conditions, including symptoms due to elevated blood pressure.

The present disclosure provides devices, systems and methods relating to prosthetic valves configured to be able to change from a compressed configuration to an expanded configuration. The prosthetic valve can be in the compressed configuration while being delivered to a target site and can be in the expanded configuration at the target site. The compressed configuration can be more compact. A prosthetic valve can have a reduced profile in the compressed configuration. For example, a width of the prosthetic valve in the compressed configuration can be narrower than that of the expanded configuration. In some embodiments, the prosthetic valve can have an elongate configuration while compressed. Transformation from a compressed configuration to an expanded configuration can comprise expansion along one or more width directions. In some embodiments, the transformation can comprise radial expansion of one or more portions of the prosthetic valve such that the expanded configuration has a width dimension larger than that in the compressed configuration.

While in the expanded configuration, the prosthetic valve can alternate between an open and a closed state, allowing fluid flow therethrough in the open state and preventing fluid flow therethrough in the closed state. In the open state, fluid can be allowed to flow through the prosthetic valve unobstructed or substantially unobstructed. For example, natural fluid flow can be unobstructed or substantially unobstructed in the open state. In the closed state, the prosthetic valve can be closed off, thereby reducing fluid flow through the prosthetic valve as compared to that in the open state. In some embodiments, the prosthetic valve can be sealed and fluid flow through the prosthetic valve can be prevented. The prosthetic valve can act as a one-way valve to allow passage of fluid in one direction (e.g., a natural flow of blood) and prevent and/or reduce flow in the opposite direction (e.g., prevent and/or reduce backflow of the blood). For example, the solutions presented herein can prevent and/or reduce backflow of blood from the left atrium into the pulmonary veins.

The prosthetic valve can comprise a body member having a conduit extending therethrough. The body member can have a first end and a second end, where a first opening can be at the first end and a second opening can be at the second end. In the expanded configuration, the elongate member can be in a diametrical orientation. In the diametrical orientation, the elongate member can be positioned over the first end of the body member. While the elongate member is in the diametrical orientation, and while the prosthetic valve is in the closed state, the covering can be configured to be positioned over and in contact with the first end of the body member such that the covering can close off, including seal, the first opening at the first end. Fluid can be prevented from flowing into the body member through the first opening. In the open state, at least a portion of the covering can be bent away from the first end of the body member such that at least a portion of the covering is spaced away from the body member, enabling fluid to flow through the first opening at the first end. In the open state, fluid can flow into the body member through the second opening, through the conduit, and out of the first end of the body member through the first opening.

In a compressed configuration, the elongate member can have a longitudinal orientation. The elongate member can have an orientation similar to or the same as the longitudinal axis of the body member. The body member can have a lateral dimension smaller than that in the expanded configuration. The lateral dimension can extend along an axis perpendicular or substantially perpendicular to that of longitudinal axis of the body member. In the compressed configuration, the covering can be folded. For example, the covering can be in a rolled configuration. One end of the elongate member, such as a first end of the elongate member, can be coupled to the first end of the body member. In some embodiments, the elongate member can be pivoted around its first end to be rotated relative to the body member and transform the elongate member between the longitudinal orientation and the diametrical orientation.

Prosthetic valves as described herein can be configured to assume a compressed configuration while within a delivery catheter, facilitating delivery of the prosthetic valves to a target site using minimally invasive transcatheter delivery techniques. The prosthetic valves can assume an expanded configuration after release from the delivery catheter. In some embodiments, the prosthetic valve can comprise shape memory material. For example, the body member and the covering can comprise shape memory material such that after release from constraint of the delivery catheter, the body member and covering can expand and/or unfold to assume the expanded configuration. The elongate member can rotate from the longitudinal orientation of the compressed configuration to the diametrical orientation of the expanded configuration. The prosthetic valves can be positioned at the target site in the expanded configuration.

The prosthetic valves can be configured to be implanted at various locations around and/or within the heart, including at least partially within the pulmonary veins, coronary sinus, superior vena cava, inferior vena cava, tricuspid valve, mitral valve, and/or aortic valve. The prosthetic valves can be positioned within various other lumens in the heart or body to prevent backflow of blood or other fluids.

Although the prosthetic valves are primarily described herein as being used for controlling blood flow into and/or within the heart, it will be understood that the use of the prosthetic valves are not so limited. The prosthetic valves can be positioned within any number of vessels, channels, valves, and/or chambers to improve control of fluid flow therethrough.

The term “associated with” is used herein according to its broad and ordinary meaning. For example, where a first feature, element, component, device, or member is described as being “associated with” a second feature, element, component, device, or member, such description should be understood as indicating that the first feature, element, component, device, or member is physically coupled, attached, or connected to, integrated with, embedded at least partially within, or otherwise physically related to the second feature, element, component, device, or member, whether directly or indirectly.

Reference herein to “catheters” and/or “delivery catheters” can refer or apply generally to any type of elongate tubular delivery device comprising an inner lumen configured to slidably receive instrumentation, such as for positioning within an atrium or coronary sinus, including for example delivery sheaths and/or cannulas.

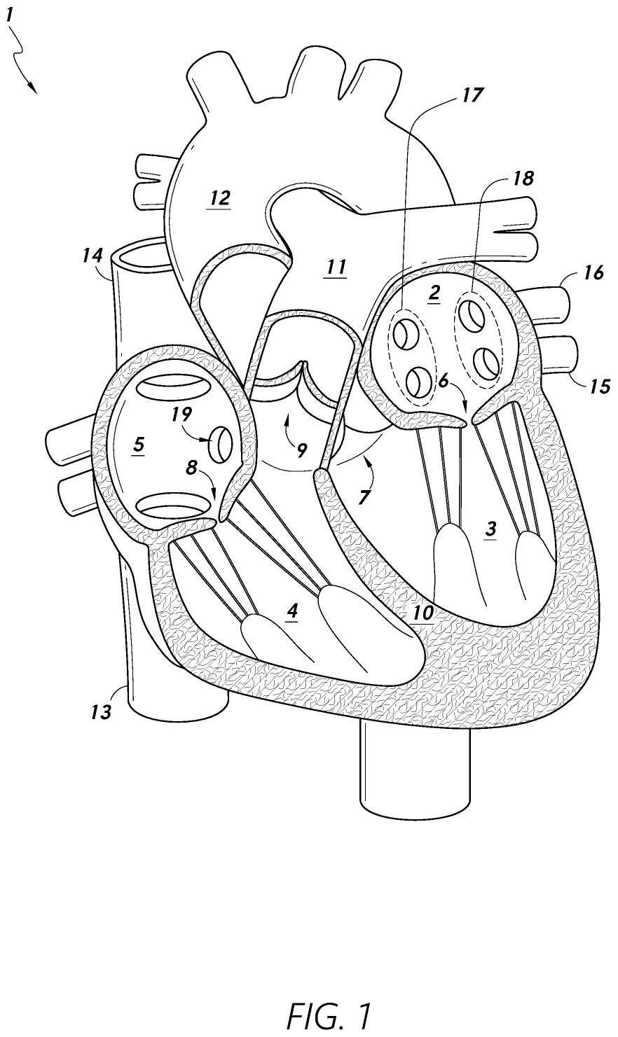

FIG. 1 is a cross-sectional view of an example heart 1 having various features/anatomy relevant to certain aspects of the present inventive disclosure. The heart 1 includes four chambers, namely the left ventricle 3, the left atrium 2, the right ventricle 4, and the right atrium 5. A wall of muscle, referred to as the septum 10, separates the left-side chambers from the right-side chambers. For example, an atrial septum wall portion separates the left atrium 2 from the right atrium 5, whereas a ventricular septum wall portion separates the left ventricle 3 from the right ventricle 4. Blood flow through the heart 1 is at least partially controlled by four valves, the mitral valve 6, aortic valve 7, tricuspid valve 8, and pulmonary valve 9. The mitral valve 6 separates the left atrium 2 and the left ventricle 3 and controls blood flow therebetween. The aortic valve 7 separates and controls blood flow between the left ventricle 3 and the aorta 12. The tricuspid valve 8 separates the right atrium 5 and the right ventricle 4 and controls blood flow therebetween. The pulmonary valve 9 separates the right ventricle 4 and the pulmonary artery 11, controlling blood flow therebetween. The mitral valve 6 separates the left atrium 2 from the left ventricle 3. The aortic valve 7 separates the left ventricle 3 from the aorta 12.

The four valves of the heart aid the circulation of blood in the heart. The tricuspid valve 8 generally closes during ventricular contraction (i.e., systole) and opens during ventricular expansion (i.e., diastole). The pulmonary valve 9 separates the right ventricle 4 from the pulmonary artery 11 and generally is configured to open during systole so that blood may be pumped toward the lungs from the right ventricle 4, and close during diastole to prevent blood from leaking back into the right ventricle 4 from the pulmonary artery. The mitral valve 6 may generally be configured to open during diastole so that blood in the left atrium 2 can flow into the left ventricle 3, and close during diastole to prevent blood from leaking back into the left atrium 2. The aortic valve 7 is configured to open during systole to allow blood leaving the left ventricle 3 to enter the aorta 12, and close during diastole to prevent blood from leaking back into the left ventricle 3.

Deoxygenated blood enters the right atrium 5 through the inferior 13 and superior 14 venae cavae. The right side of the heart then pumps this deoxygenated blood into the pulmonary arteries around the lungs. There, fresh oxygen enters the blood stream, and the blood moves to the left side of the heart via a network of pulmonary veins ultimately terminating at the left atrium 2, as shown. The primary roles of the left atrium 2 are to act as a holding chamber for blood returning from the lungs (not shown) and to act as a pump to transport blood to other areas of the heart. The left atrium 2 receives oxygenated blood from the lungs via the pulmonary veins 15, 16. The ostia 17, 18 of the pulmonary veins 15, 16 are generally located at or near posterior left atrial wall of the left atrium 2. The oxygenated blood that is collected from the pulmonary veins 15, 16 in the left atrium 2 enters the left ventricle 3 through the mitral valve 6.

Surrounding the ventricles (3, 4) are a number of arteries that supply oxygenated blood to the heart muscle and a number of veins that return the blood from the heart muscle to the right atrium 5 via the coronary sinus. The coronary sinus is a relatively large vein that extends generally around the upper portion of the left ventricle 3 and provides a return conduit for blood returning to the right atrium 5. The coronary sinus terminates at the coronary ostium 19, through which the blood enters the right atrium 5.

Heart failure (HF) generally occurs when the heart is unable to pump sufficiently to maintain blood flow to meet the body's needs. In some cases, changes in cardiac pressures are associated with heart failure, such as pressure elevation in the left atrium. Elevated pressure in the lungs and/or one or more chambers of the heart can impede normal blood flow through the heart. Elevated pressure in the lungs and/or dysfunction in the aortic valve can result in undesired flow of blood back into the left atrium. Dysfunction of the mitral valve and/or tricuspid valve (e.g., mitral valve regurgitation and/or stenosis) can contribute to elevated left atrial pressure and/or right atrial pressure, respectively. Furthermore, there may be a relatively strong correlation between increases in left atrial pressure and pulmonary congestion. As such, in some instances, blood can flow back into the pulmonary veins from the left atrium to cause blood build-up in the lungs. Symptoms can include shortness of breath during exertion, fatigue, chest pain, fainting, abnormal heartbeat, and swelling of the legs and feet.

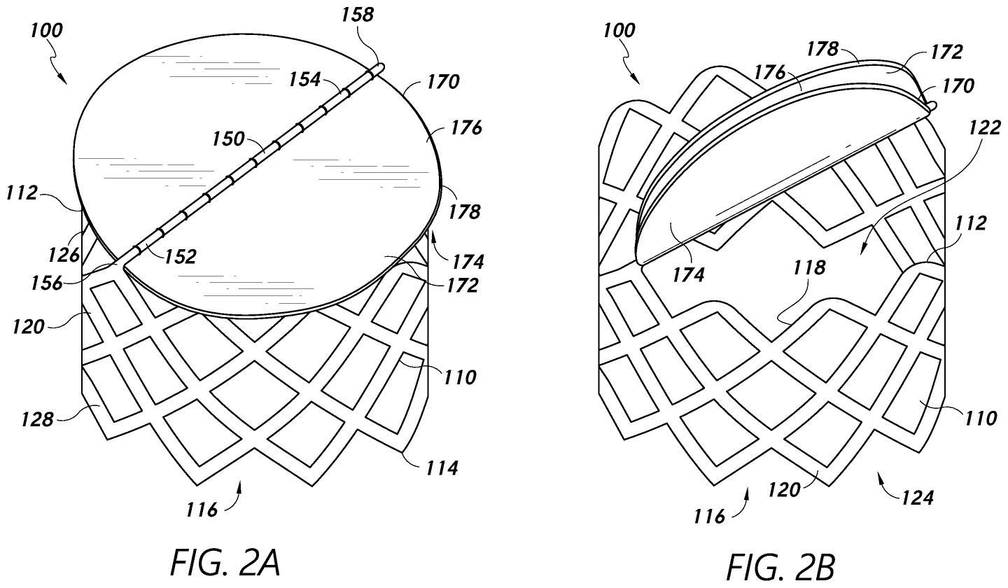

FIGS. 2 through 4 show various views of an example of a prosthetic valve 100. The prosthetic valve 100 can assume an expanded configuration and a compressed configuration. FIGS. 2A and 2B are perspective views of the prosthetic valve 100 in the expanded configuration. The prosthetic valve 100 can be configured to alternate between an open state and a closed state in the expanded configuration. In the open state, the prosthetic valve 100 can be configured to permit fluid flow therethrough. In the closed state, the prosthetic valve 100 can be configured to prevent fluid flow therethrough. FIG. 2A shows the prosthetic valve 100 in a closed state and FIG. 2B shows the prosthetic valve 100 in an open state. In some embodiments, the prosthetic valve 100 can act as a one-way valve to allow passage of blood in one direction in the open state, and, in the closed state, prevent flow in the opposite direction. For example, in the open state, the prosthetic valve can permit flow therethrough of blood along a natural flow path. In the closed state, the prosthetic vale can prevent backflow of the blood against the flow of the natural flow path. As described herein, the prosthetic valve 100 can be configured prevent backflow of blood, including from the left atrium into the pulmonary veins, and/or from the right atrium into the coronary sinus, superior vena cava, and/or inferior vena cava. In some embodiments, the prosthetic valve 100 can be configured to prevent backflow of blood from the right ventricle into the right atrium, from the left ventricle into the left atrium and/or from the lungs into the left ventricle.

Referring to FIGS. 2A and 2B, the prosthetic valve 100 can comprise a body member 110, an elongate member 150 associated with the body member 110, and a covering 170 coupled to the elongate member 150. As described herein, in some embodiments, the elongate member 150 and the body member 110 can be integrally formed. Alternatively, the elongate member 150 can be coupled to the body member 110. The body member 110 can comprise a first end 112 and a second end 114. The body member 110 can define a conduit 116 extending therethrough, for example such that the first end 112 can comprise a first opening 122 and the second end 114 can comprise a second opening 124. The body member 110 can comprise an inner surface 118 oriented toward the conduit 116, and an outer surface 120. The outer surface 120 can be configured to be oriented toward tissue at the target tissue site. In the open state, fluid can flow through the conduit 116, such as through the second opening 124 at the second end 114, through the conduit 116 from the second end 114 to the first end 112, and through the first opening 122 at the first end 112. In some embodiments, the body member 110 can comprise a cylindrical configuration when the prosthetic valve 100 is in the expanded configuration. For example, the body member 110 can form a cylindrical shape in the expanded configuration. In some embodiments, while the prosthetic valve 100 is in the expanded configuration, the conduit 116 can comprise a cylindrical shape. In some embodiments, while the prosthetic valve 100 is in the expanded configuration, the first opening 122 at the first end 112 of the body member 110 and the second opening 124 at the second end 114 can each comprise a circular shape.

In some embodiments, the body member 110 can comprise a wired configuration. For example, the body member 110 can be formed at least in part using shape memory wires. In some embodiments, the body member 110 can comprise self-expandable wire frame configured to self-expand upon release from constraint of a delivery catheter in which the prosthetic valve resides during advancement to the target site. In some embodiments, the body member 110 can comprise shape memory wires forming a cylindrical shape.

The elongate member 150 can comprise a first end portion 152 and a second end portion 154. In some embodiments, the elongate member 150 can comprise a linear or substantially linear configuration. As shown in FIGS. 2A and 2B, in some embodiments, the elongate member 150 can be integral with the body member 110. The elongate member 150 and the body member 110 can be one piece. For example, the elongate member 150 can extend from the first end 112 of the body member 110. In some embodiments, the body member 110 and the elongate member 150 can comprise a shape memory material. The body member 110 and the elongate member 150 can be shape set such that the elongate member 150 can change from a longitudinal orientation to a diametrical orientation while the prosthetic valve 100 changes from a compressed configuration to an expanded configuration. The prosthetic valve 100 can be shape set such that the elongate member 150 can be across the first end 112 of the body member 110 when the prosthetic valve 100 is in the expanded configuration. For example, during transformation of the prosthetic valve 100 from the compressed to the expanded configuration, the elongate member 150 can fold down from the longitudinal orientation to be positioned across the first end 112 of the body member 110, assuming the diametrical orientation. The elongate member 150 can be shape set to move from the longitudinal orientation to the diametrical orientation.

Alternatively, the elongate member 150 and the body member 110 can be two distinct components that are coupled to one another. The first end portion 152 of the elongate member 150 can be coupled to the body member 110. For example, the elongate member 150 can be coupled to a first end portion 126 of the body member 110, such as a first end 112 of the body member 110. In some embodiments, the first end portion 152 of the elongate member 150 can be rotatably coupled to the body member 110. For example, the first end portion 152 of the elongate member 150 can be rotatably coupled to the first end 112 of the body member 110 such that the elongate member 150 can pivotally rotate around its first end portion 152 relative to the first end 112 of the body member 110. As described in further detail herein, during transformation of the prosthetic valve 100 from a compressed configuration to an expanded configuration, the elongate member 150 can be rotated relative to the body member 110 to position the elongate member 150 from a longitudinal orientation to a diametrical orientation. In some embodiments, the prosthetic valve 100 can comprise a hinge connector configured to rotatably couple the first end portion 152 of the elongate member 150 and the body member 110.

As shown in FIGS. 2A and 2B, while the prosthetic valve is in the expanded configuration, the elongate member 150 can assume a diametrical orientation. The elongate member 150 can comprise at least a portion positioned over the first end 112 of the body member 110. The elongate member 150 can comprise at least a portion which extends along a plane parallel or substantially parallel to a plane along which the first opening 122 extends. For example, the first opening 122 can extend along a plane perpendicular or substantially perpendicular to a longitudinal axis of the body member 110. The longitudinal axis of the body member 110 can extend along a dimension extending between the first end 112 and second end 114 of the body member 110. The elongate member 150 can extend along one or more planes perpendicular or substantially perpendicular to the longitudinal axis of the body member 110. In some embodiments, the elongate member 150 can be positioned across a diameter of the first end 112 of the body member 110, including a diameter of the first opening 122. The first end portion 152 and second end portion 154 of the elongate member 150 can be at opposing positions on the first end 112 of the body member 110. For example, the first end portion 152 and the second end portion 154 of the elongate member 150 can be coupled to diametrically opposing portions of the first end 112 of the body member 110.

In some embodiments, while the elongate member 150 is in the diametrical orientation, the second end portion 154 of the elongate member 150 can be coupled to the body member 110, including the first end portion 126, such as the first end 112 of the body member 110. For example, the first end portion 152 of the elongate member 150 can be associated with the first end 112 of the body member 110 at a first position on the first end 112. The second end portion 154 of the elongate member 150 can be coupled to the first end 112 of the body member 110 at a second position on the first end 112. Any number of coupling mechanisms can be used to couple the second end portion 154 of the elongate member 150 to the first end 112 of the body member.

Referring again to FIGS. 2A and 2B, while the prosthetic valve 100 is in the expanded configuration, the covering 170 can be in an unfolded configuration. While the prosthetic valve 100 is in the expanded configuration and in the closed state, the covering 170 can be positioned over the first end 112 of the body member 110 to close off fluid flow through the prosthetic valve 100. In the closed state, the prosthetic valve 100 can be configured to reduce or eliminate fluid flow therethrough. The prosthetic valve 100 can be configured to prevent fluid flow into the first end 112 of the body member 110 and thereby preventing fluid flow through the conduit 116. For example, the covering 170 can comprise an orientation perpendicular or substantially perpendicular to the longitudinal axis of the body member 110. The covering 170 can comprise at least a portion parallel or substantially parallel to the plane in which the first end 112 and the first opening 122 extends. The covering 170 can extend across the first end 112 of the body member 110, for example completely covering the first end 112 of the body member 110, including the first opening 122 at the first end 112. Covering the first opening 122 can facilitate closing off of the first end 112 of the body member 110 against fluid flow into the first end 112. For example, covering 170 can seal the first end 112 in the closed state.

The covering 170 can comprise a first surface 172 and a second surface 174. While positioned over the first end 112 of the body member 110, the first surface 172 can be oriented away from the first end 112 and the second surface 174 can be oriented toward the first end 112. In the closed state, the second surface 174 can be over and in contact with the first end 112 of the body member 110. For example, at least partially while the prosthetic valve 100 is at a target site, fluid can be in contact with the second surface 174 of the covering 170 and can exert pressure upon the covering 170 to press the covering 170 against the first end 112 of the body member 110. Maintaining contact between the covering 170 and the first end 112 of the body member 110 can reduce or eliminate fluid flow into the first end 112 of the body member 110, thereby facilitating closing off of the first opening 122 at the first end 112 of the body member 110. In some embodiments, the first opening 122 can be sealed and fluid flow through the prosthetic valve 100 can be prevented. In some embodiments, pressure exerted by fluid upon the elongate member 150 and/or the covering 170 while the covering 170 is positioned over the first end 112 can facilitate coupling of the second end 158 of the elongate member to the first end 112. For example, pressure exerted upon the elongate member 150 and/or the covering 170 can facilitate the second end 158 clicking and/or snapping into place.

In some embodiments, the covering 170 can have a lateral dimension, such as a width, larger than that of the first end 112 of the body member 110 to facilitate secure positioning of the covering 170 over the first end 112. For example, the covering 170 can comprise portions which extend along one or more planes parallel or substantially parallel to the plane in which the first end 112 of the body member 110 extends and which have dimensions larger than that of corresponding portions of the first end 112 over which they are positioned. In some embodiments, the covering 170 comprises portions which extend laterally beyond corresponding portions of the first end 112 over which they are positioned. For example, the covering 170 can comprise portions which extend along a direction perpendicular or substantially perpendicular to the longitudinal axis of the body member 110, and beyond the outer surface 120 of the body member 110. At least a portion of an edge portion 176 of the covering 170 can extend laterally beyond a corresponding portion of the first end 112 to facilitate securely sealing of the first end 112, thereby closing off the prosthetic valve 100, preventing or reducing fluid flow through the prosthetic valve 100.

In some embodiments, the covering 170 can be coupled to the elongate member 150 along a longitudinal dimension of the elongate member 150. The covering 170 can be coupled to the elongate member 150 along an entire or a portion of the length of the elongate member 150. In some embodiments, sutures can extend through a portion of the covering 170, such as at least a portion of a length and/or a width of the covering 170, to couple the covering 170 to the elongate member 150. For example, sutures extending through an entire or substantially entire length and/or entire or substantially entire width of the covering 170 can couple the covering 170 to the elongate member 150 along an entire or substantially entire length of the elongate member 150. Other methods can be used to couple the covering 170 to the elongate member 150, including for example lamination, adhesion, and/or insertion between and/or within portions of the covering 170.

As described herein, in some embodiments, the first end 112 of the body member 110 can comprise a circular or substantially circular shape. In some embodiments, the covering 170 can comprise a circular or substantially circular shape. The circular or substantially circular covering 170 can comprise a size larger than that of the first end 112, for example comprising a diameter larger than that of the first end 112. An edge portion 176 of the covering 170 can extend beyond the first end 112. As shown in FIGS. 2A and 2B, in some embodiments, the covering 170 can be a single leaflet. The leaflet can comprise a circular shape. The leaflet can be coupled to the elongate member 150 along a diameter of the leaflet. The leaflet can comprise a size larger than that of the first end 112 of the body member 110 to facilitate securely sealing the first end 112 when the leaflet is in contact with the first end 112. As described in further detail herein, in some embodiments, the covering 170 can comprise more than one leaflet.

One or both of the first end 112 and the second end 114 of the body member 110 can comprise an atraumatic configuration. The atraumatic configuration can be configured to prevent abrasion and/or damage to surrounding tissue at the target site. In some embodiments, the first end 112 can comprise a rounded configuration. As described herein, the body member 110 can comprise a wired configuration. In some embodiments, an atraumatic first end can comprise curved wire portions to form rounded portions at the first end 112. In some embodiments, an atraumatic second end can comprise curved wire portions to form rounded portions at the second end 114.

FIG. 2B shows the prosthetic valve 100 in the expanded configuration and in the open state. While the prosthetic valve 100 is in the open state, the elongate member 150 can be positioned across the first end 112 of the body member 110. At least a portion of the covering 170 can be configured to be spaced from the first end 112 of the body member 110 such that fluid can flow through the prosthetic valve 100. In the open state, fluid can flow through the conduit 116 of the body member 110 from the first end 112 to the second end 114 of the body member 110. In some embodiments, portions of the covering 170 can be parallel or substantially parallel to a longitudinal axis of the body member 110.

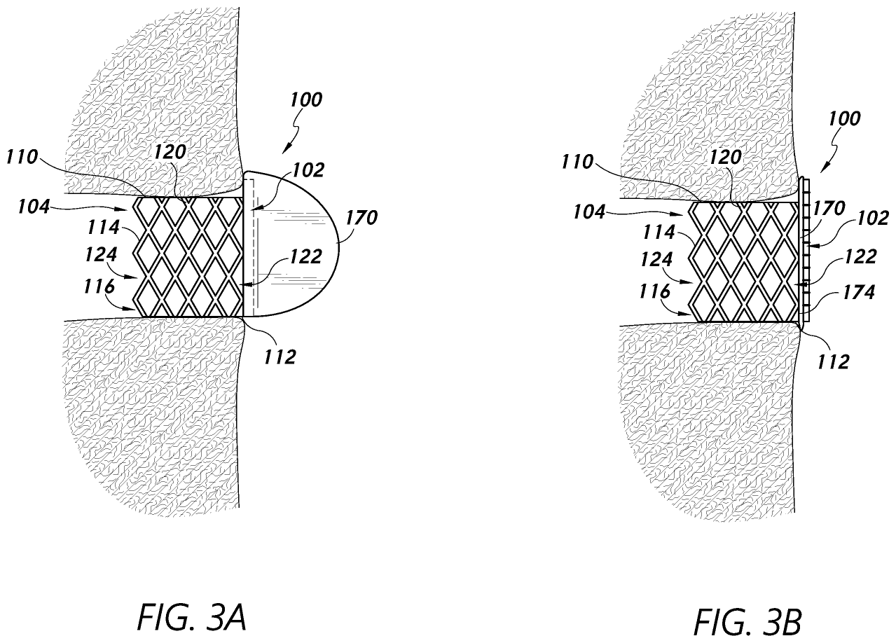

FIGS. 3A and 3B are side views showing the prosthetic valve 100 positioned at a target site. At least a portion of the body member 110 can be positioned within a vessel, lumen, valve and/or chamber. For example, at least a portion of the outer surface 120 of the body member 110 can be oriented toward and in contact with tissue of the vessel, lumen, valve and/or chamber. The portion of the outer surface 120 can be adjacent to and in contact with the tissue to facilitate stable positioning of the prosthetic valve 100 at the target site and/or prevent fluid leakage between the prosthetic valve 100 and the target tissue.

FIG. 3A shows the prosthetic valve 100 in the open state and FIG. 3B shows the prosthetic valve 100 in the closed state. In the open state, the prosthetic valve 100 can be positioned at the target site such that fluid can flow through the prosthetic valve 100, including from a second end 104 to a first end 102 of the prosthetic valve 100. For example, fluid can flow into the body member 110 through the second opening 124 at the second end 114 of the body member 110, through the conduit 116, and out of the body member 110 through the first opening 122 at the first end 112 of the body member 110. In the closed state, the prosthetic valve 100 can prevent fluid from entering the first end 102 of the prosthetic valve, thereby preventing fluid flow through the prosthetic valve 100 in a direction from the first end 102 to the second end 104. The prosthetic valve 100 can prevent fluid from entering the body member 110 through the first opening 122 at the first end 112 of the body member 110.

FIG. 3A shows at least a portion of the covering 170 positioned away from the first end 112 of the body member 110. In the open state, fluid flowing through the first opening 122 can exert pressure upon the second surface 174 of the covering 170 to push at least a portion of the covering 170 away from the first end 112. The covering 170 can be folded back toward itself due to force exerted upon the second surface 174. For example, the second surface 174 is shown in FIG. 3A and the covering 170 is folded back upon itself such that portions of the first surface 172 are oriented toward one another.

FIG. 3B shows the covering 170 positioned over the first end 112 of the body member 110. While in the covering 170 is positioned over the first end 112, fluid can be in contact with the first surface 172 of the covering 170 to push the covering 170 against the first end 112 of the body member 110 to close off the first opening 122. Closing off of the first opening 122 of the body member 110 can thereby reduce or prevent fluid from flowing through the first opening 122 and entering the body member 110, for example sealing the first opening 122.

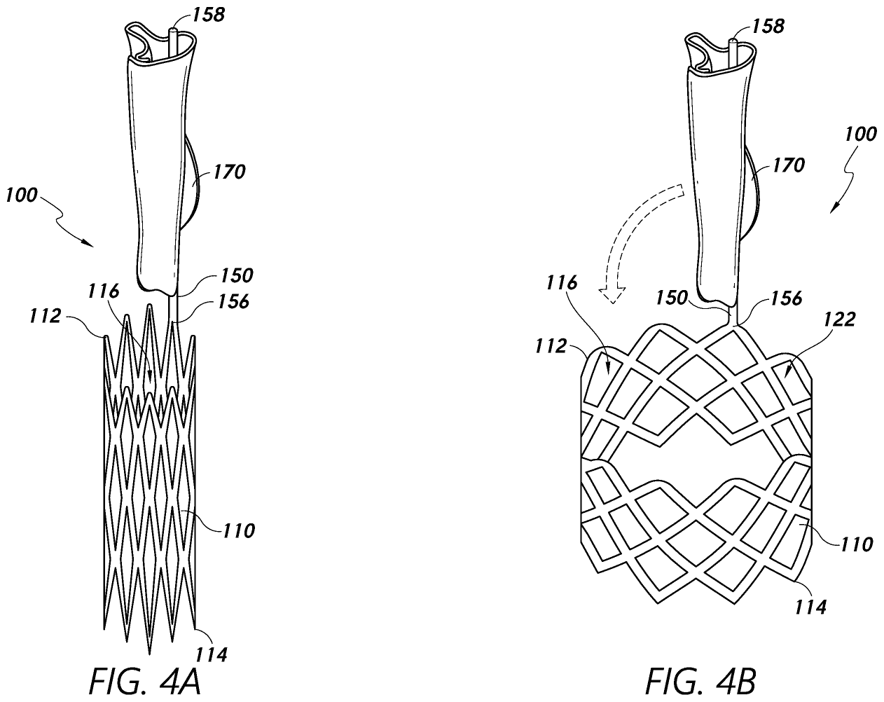

FIG. 4A is a perspective view of the prosthetic valve 100 in a compressed configuration, and FIG. 4B is a perspective view of the prosthetic valve 100 in an expanded configuration. While the prosthetic valve 100 is in the compressed configuration, a lateral dimension, such as a width of the body member 110 can be smaller than that in the expanded configuration. The width of the prosthetic valve 100 in the compressed configuration can be predetermined to facilitate delivery through a delivery catheter. A length of the prosthetic valve 100 can be the same as or longer in the compressed configuration as that in the expanded configuration. The width can be a dimension extending along a direction perpendicular or substantially perpendicular to the longitudinal axis of the body member 110. The length can be a dimension extending along a direction parallel or substantially parallel to the longitudinal axis of the body member 110. In some embodiments, the body member 110 can expand along one or more lateral directions to transform from the compressed to the expanded configuration. The body member 110 can extend radially to assume a configuration having a larger width. The width of the body member 110 can be predetermined based on anatomical dimensions at a target site.

The elongate member 150 is shown in a longitudinal orientation in both figures. As described herein, the elongate member 150 can be configured to change between the longitudinal orientation and a diametrical orientation. In some embodiments, the longitudinal orientation can comprise an orientation where the body member 110 and the elongate member 150 comprise a same or similar orientation. For example, in the longitudinal orientation a longitudinal axis of the elongate member 150 can have the same or similar orientation as the longitudinal axis of the body member 110. In the longitudinal orientation, the second end 158 of the elongate member 150 can be positioned away from the first end 112 of the body member 110. In some embodiments, in the longitudinal orientation, the longitudinal axis of the elongate member 150 can be parallel or substantially parallel to that of the body member 110.

The elongate member 150 can assume the diametrical orientation while the prosthetic valve 100 is in the expanded configuration. For example, the elongate member 150 can pivot around the first end 156 to rotate from the longitudinal orientation to the diametrical orientation. In the diametrical configuration, the second end 158 of the elongate member 150 can be in contact with the first end 112 of the body member 110. The elongate member 150 can extend along a plane parallel or substantially parallel to that in which the first end 112 extends. In some embodiments, the longitudinal orientation is perpendicular or substantially perpendicular to the diametrical orientation. In some embodiments, in the diametrical orientation, the longitudinal axis of the elongate member 150 is perpendicular or substantially perpendicular to the longitudinal axis of the body member 110.

In FIGS. 4A and 4B, the covering 170 is shown as being folded. The covering 170 can be folded during delivery to a target site. The covering 170 can be in a folded configuration prior to being released from the delivery catheter. The folded configuration can comprise a rolled configuration. For example, the covering 170 can be in a rolled configuration during delivery to the target site. The rolled configuration can comprise a longitudinal axis having the same or similar orientation as the longitudinal axis of the body member 110. In some embodiments, the rolled configuration can comprise a longitudinal axis parallel or substantially parallel to that of the body member 110. After being released from the delivery catheter, the covering 170 can assume the unfolded configuration. The covering 170 can unfold as it is released from the delivery catheter, for example unrolling to assume a fully expanded state. In some embodiments, the covering 170 can self-expand upon release from the constraint of the delivery catheter, unfolding to assume a planar or substantially planar configuration.

In some embodiments, the prosthetic valve 100 can comprise shape memory material. In some embodiments, the prosthetic valve can be shape set to assume the expanded configuration after release from the constraint of the delivery catheter. For example, after being released from the constraint of the delivery catheter, the body member 110 can radially expand along one or more lateral dimensions to assume a wider state. For example, the body member 110 can assume a wider cylinder. The covering 170 can unroll to assume the unfolded configuration. The elongate member 170 can rotate around its first end 156 to be positioned over the first end 112 of the body member 110, extending across the first opening 122. In some embodiments, the body member 110 and the elongate member 150 can be shape set such that the elongate member 150 can move from the longitudinal orientation to the diametrical orientation in the transformation of the prosthetic valve 100 from the compressed configuration to the expanded configuration.

In some embodiments, a covering can comprise more than one unitary covering member. In some embodiments, a covering can comprise two separate covering members. For example, a first and second covering, such as a first leaflet and a second leaflet, can each be coupled to an elongate member. Suturing, lamination, adhesion and/or insertion between and/or within portions of the respective covering can be used to couple the first and second coverings to the elongate member. In some embodiments, the first and second coverings can be rotatably coupled to the elongate member. The first and second coverings can be configured to be pivotally rotatable around the elongate member to position the coverings over the first end of the body member while the prosthetic valve in the closed state or away from the first end of the body member is in the open state.

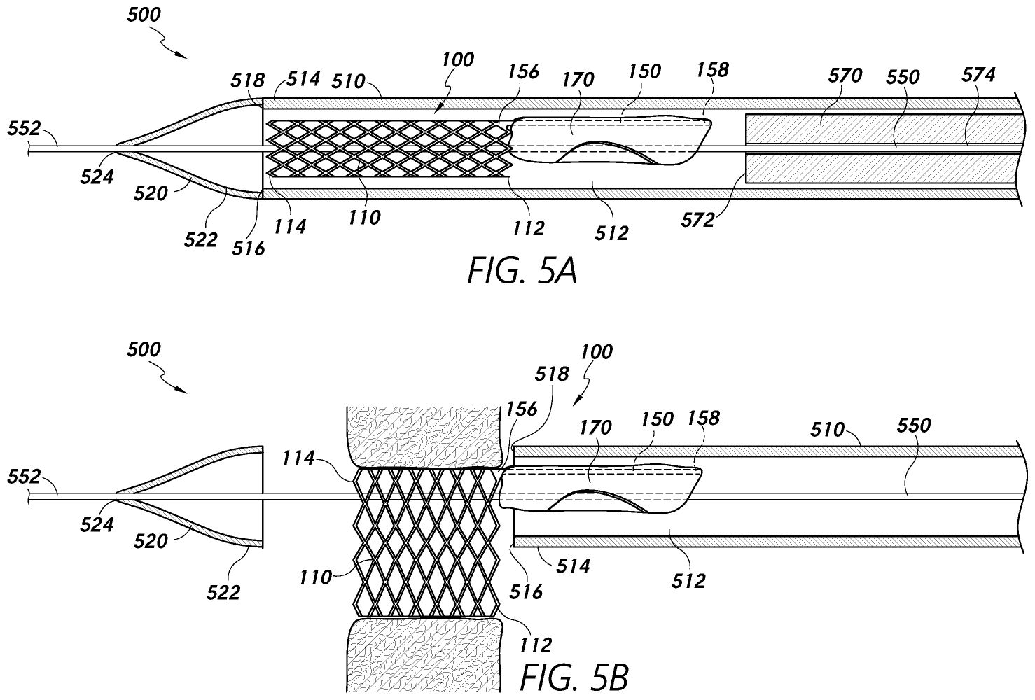

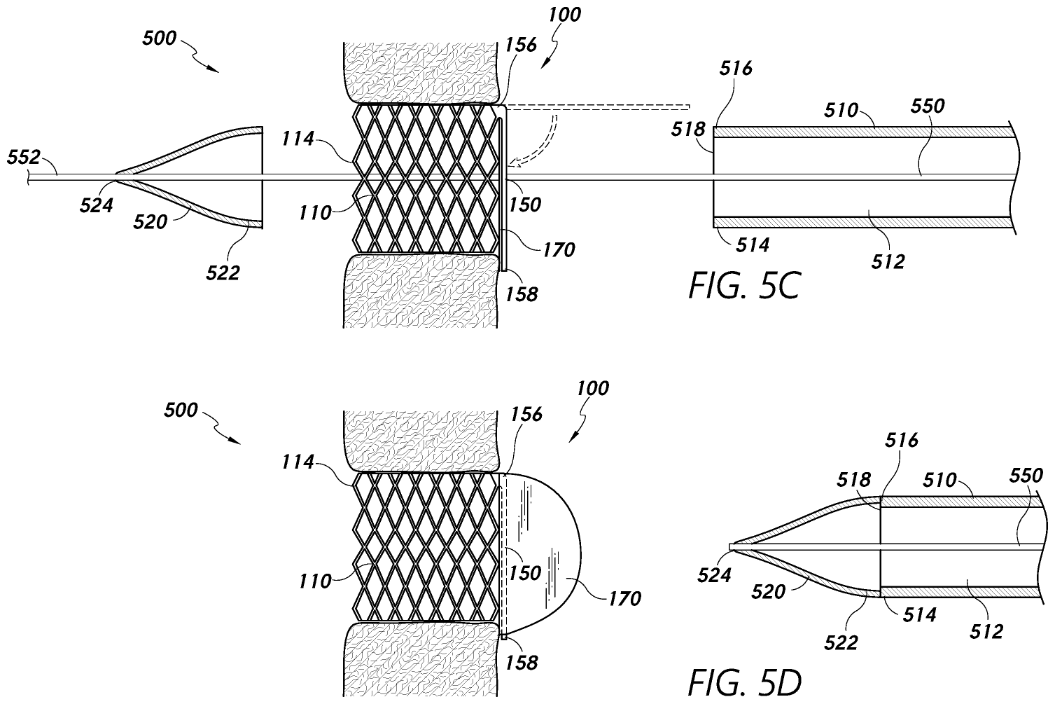

FIGS. 5A, 5B, 5C and 5D are cross-sectional side views of an example of a prosthetic valve delivery system 500 positioning the prosthetic valve 100 at a target site. Referring to FIG. 5A, the delivery system 500 can comprise a delivery catheter 510 comprising a delivery lumen 512 extending therethrough. The prosthetic valve 100 can be positioned within the delivery lumen 512, such as within the delivery lumen 512 in a distal portion 514 of the delivery catheter 510. The delivery system 500 can comprise a guide wire 550 extending through the delivery lumen 512. The prosthetic valve 100 can be positioned around the guide wire 550 while within the distal portion 514 the delivery catheter 510. In some embodiments, the prosthetic valve 100 and/or the delivery catheter 510 can be advanced along the guide wire 550 to a target position within a patient, such as to a target position within the heart. For example, the prosthetic valve 100 can be pre-loaded within the distal portion 514 of the delivery catheter 510 and can be advanced along with the distal portion 514 of the delivery catheter 510 along the guide wire 550 to the target position. In some embodiments, the distal portion 514 of the delivery catheter 510 can be advanced along the guide wire 550 to the target position prior to advancing the prosthetic valve 100 along the guide wire 550 within the delivery lumen 512 to the target position.

As described herein, the prosthetic valve 100 can be in a compressed configuration while within the delivery catheter 510. The elongate member 150 can be in a longitudinal orientation. In FIG. 5A, the first end 112 of the body member 110 is shown as being oriented proximally and the second end 114 of the body member 110 is shown as being oriented distally.

A distal tip portion 520 can be associated with a distal end 516 of the delivery catheter 510. The distal tip portion 520 can be configured to facilitate navigation of the delivery catheter 510 through tortuous anatomical pathways. The distal tip portion 520 can comprise a decrease in size from a proximal end 522 to a distal end 524 of the distal tip portion 520, for example tapering from the proximal end 522 to the distal end 524. In some embodiments, the distal tip portion 520 can comprise a conical shape, the smaller end of the conical shape being oriented distally. The proximal end 522 of the distal tip portion 520 can be coupled to the distal end 516 of the delivery catheter 510. The distal tip portion 520 can be reversibly separable from the distal end 516 to facilitate release of the prosthetic valve 100 through the distal opening 518 at the distal end 516. The prosthetic valve 100 can be translated distally relative to the delivery catheter 510 to release the prosthetic valve 100 from the distal end 516 of the delivery catheter 510. In some embodiments, the delivery system 500 can comprise a pusher shaft 570. The pusher shaft 570 can comprise a distal end 572 configured to contact the prosthetic valve 100 to push the prosthetic valve 100 distally relative to the delivery catheter 510. The pusher shaft 570 can push the prosthetic valve 100 distally such that the prosthetic valve 100 can exit the delivery catheter 510 through the distal end 516 of the delivery catheter 510, such as through the distal end 524 of the distal tip portion 520. The prosthetic valve 100 can be passed through the distal opening 518 at the distal end 516 of the delivery catheter 510.

In some embodiments, the pusher shaft 570 can contact the second end 158 of the elongate member 150 to translate the prosthetic valve 100 distally. A distal end 572 of the pusher shaft 570 can contact the second end 158. In some embodiments, a pusher shaft can comprise a configuration to push the second end 158 of the elongate member 150 and/or one or more other portions of the prosthetic valve 100. In some embodiments, a pusher shaft 570 can be configured to contact at least a portion of the first end 112 of the body member 110. The pusher shaft 570 can comprise a guide wire lumen 574 extending therethrough. The guide wire 550 can extend through the guide wire lumen 574 such that the pusher shaft 570 can be advanced and/or retracted along the guide wire 550. It will be understood that the orientation of the prosthetic valve 100 within the delivery catheter 510 can depend on the orientation of the prosthetic valve 100 at the target site. In some embodiments, the first end 112 of the body member 110 can be oriented distally and the second end 114 of the body member 110 can be oriented proximally. The pusher shaft 570 can be configured to contact the second end 114 of the body member 110 to translate the prosthetic valve 100 distally.

The prosthetic valve 100 can assume an expanded configuration after the prosthetic valve 100 exits the distal end 516 of the delivery catheter 510. In some embodiments, the prosthetic valve 100 can comprise a shape memory material such that the prosthetic valve 100 can be shape set to assume the expanded configuration after being released from the constraint of the delivery catheter 510. The pusher shaft 570 can push the prosthetic valve 100 through the distal opening 518. Referring to FIG. 5B, the body member 110 can expand from the compressed configuration after it is released from the delivery catheter 510. The body member 110 can be in an expanded configuration after release through the distal opening 518. The distal tip portion 520 can be separated from the delivery catheter 510 to facilitate advancing the prosthetic valve 100 through the distal opening 518, for example the distal end 522 of the distal tip portion 520 decoupling from the distal end 516 of the delivery catheter 510. The body member 110 can be positioned at the target site after it is extended through the distal opening 518. For example, the body member 110 can be positioned at the target site as it is released and can expand to fit at the target site. The body member 110 can expand along one or more directions perpendicular or substantially perpendicular to its longitudinal axis to assume a configuration comprising a larger width, such as a larger diameter. The distal top portion 520 can remain distal of the prosthetic valve 100 as the prosthetic valve 100 is released.

After release from the delivery catheter 510, the covering 170 can unfold to assume an unfolded configuration. Referring to FIG. 5C, after release from the delivery catheter 510, the elongate member 150 can be configured to transform from the longitudinal orientation to a diametrical orientation. As described herein, the elongate member 150 and the body member 110 can be integrally formed and comprise shape memory material which is shape set to move the elongate member 150 from the longitudinal orientation into the diametrical orientation as the prosthetic valve 100 transforms from the compressed configuration into the expanded configuration.

FIG. 5D shows the prosthetic valve 100 positioned at a target site. As described herein, the prosthetic valve 100 can be used to control fluid flow at one or more positions within a vessel, lumen, valve and/or chamber, such as within the heart. The prosthetic valve 100 is shown in an open state in the figure. The covering 170 is shown as being spaced away from the first end of the body member 110, thereby allowing fluid to flow therethrough. The distal tip portion 520 can be retracted after the prosthetic valve 100 is at the target site. For example, the distal tip portion 520 can be retracted back through the prosthetic valve 100. The distal tip portion 520 can be retracted back toward the delivery catheter 510, for example being positioned adjacent to the distal end 516. In some embodiments, the distal tip portion 520 can be re-coupled to the distal end 516 of the delivery catheter 510. The distal tip portion 520 and the delivery catheter 510 can then be removed.

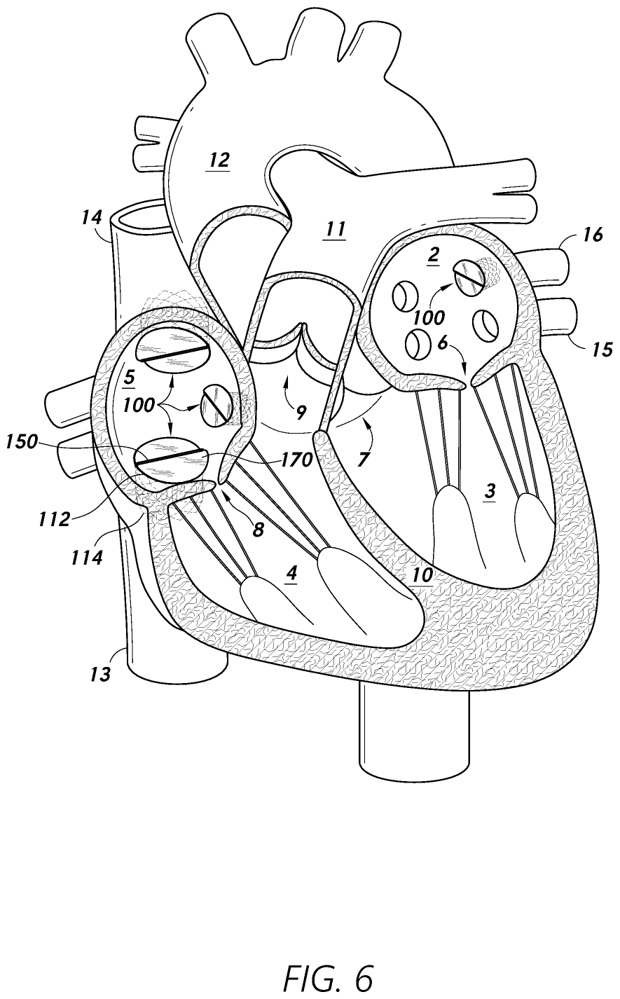

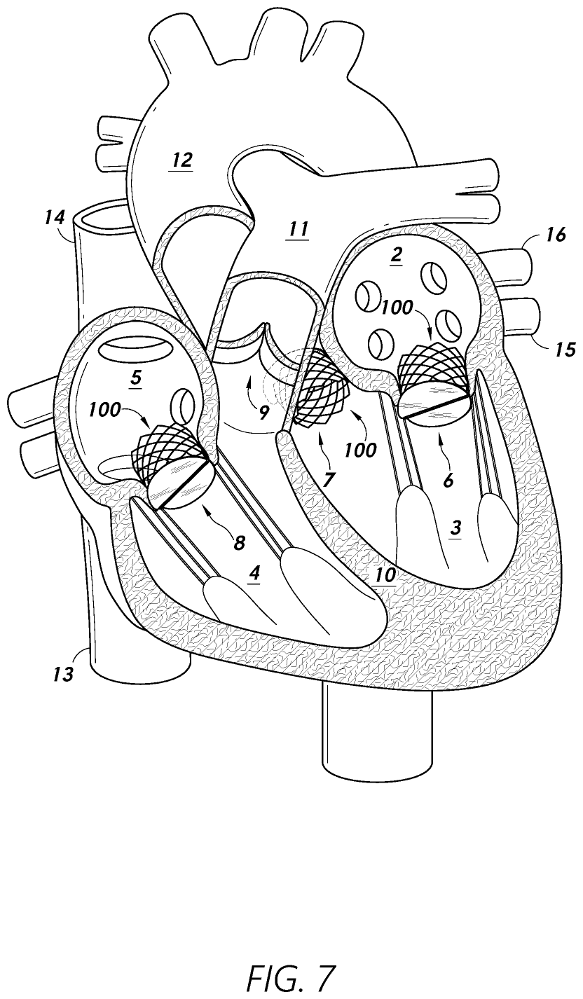

FIGS. 6 and 7 show examples of various target sites within the heart at which the prosthetic valve 100 can be positioned. Referring to FIG. 6, in some embodiments, the prosthetic valve 100 can be configured to control fluid flow from the left atrium 2 into one or more of the pulmonary veins 15, 16. In some embodiments, the prosthetic valve 100 can be configured to control fluid flow from the right atrium 5 into the coronary sinus, inferior vena cava 13 and/or superior vena cava 14. At least a portion of the prosthetic valve 100 can be positioned within one of the pulmonary veins 15, 16. In some embodiments, at least a portion of the prosthetic valve 100 can be positioned at or proximate to one of the pulmonary vein openings 17, 18 which opens into the left atrium 2. The prosthetic valve 100 can be oriented such that the first end 112 of the body member 110 is positioned closer to the left atrium 2 and the second end 114 of the body member 110 is positioned further from the left atrium 2. The prosthetic valve 100 can be oriented such that in the closed state, the prosthetic valve 100 can reduce or prevent fluid flow from the left atrium 2 into the pulmonary veins 15, 16.

In some embodiments, at least a portion of the prosthetic valve 100 can be positioned in one or more of the coronary sinus, superior vena cava 14 and inferior vena cava 13. The prosthetic valve 100 can be oriented such that in the closed state, the prosthetic valve 100 can reduce or prevent blood flow from the right atrium 5 into the coronary sinus, superior vena cava 14 or inferior vena cava 13. In the open state, the prosthetic valve 100 can allow blood flow from the coronary sinus, superior vena cava 14 or inferior vena cava 13 into the right atrium 5. At least a portion of the prosthetic valve 100 can be positioned within one or more of the coronary sinus, superior vena cava 14 and inferior vena cava 13. In some embodiments, at least a portion of a prosthetic valve 100 can be positioned at or proximate to the coronary ostium 19, an opening to the superior vena cava 14 or inferior vena cava 13 into the right atrium 5. The prosthetic valve 100 can be oriented such that the first end 112 of the body member 110 is positioned closer to the right atrium 5 and the second end 114 of the body member 110 is positioned further from the right atrium 5. For example, the second end 114 can be within the coronary sinus, superior vena cava 14 or inferior vena cava 13, while the first end 112 and the covering 170 can be in the right atrium 5.

Referring to FIG. 7, in some embodiments, the prosthetic valve 100 can be configured to control fluid flow from the right ventricle 4 into the right atrium 5, from the left ventricle 3 into the left atrium 2, or from the left ventricle 3 into the aorta 12. For example, the prosthetic valve 100 can be positioned within one or more of the tricuspid valve 8, mitral valve 6 and aortic valve 7. While positioned in the tricuspid valve 8, the prosthetic valve 100 can be oriented such that the first end 112 of the body member 110 is oriented toward the right ventricle 4 and the second end 114 of the body member 110 is oriented toward the right atrium 5. While positioned in the mitral valve 6, the prosthetic valve 100 can be oriented such that the first end 112 of the body member 110 is oriented toward the left ventricle 3 and the second end 114 of the body member 110 is oriented toward the left atrium 2. While positioned in the aortic valve 7, the prosthetic valve 100 can be oriented such that the first end 112 of the body member 110 is oriented toward the aorta 12 and the second end 114 of the body member 110 is oriented toward the left ventricle 3. The prosthetic valve 100 can be oriented such that in the closed state, the prosthetic valve 100 can reduce or prevent fluid flow from the right ventricle 4 into the right atrium 5, from the left ventricle 3 into the left atrium 2, or from the left ventricle 3 into the aorta 12. In the open state, the prosthetic valve 100 can allow blood flow from the right atrium 5 into the right ventricle 4, from the left atrium 2 into the left ventricle 3, or from the aorta 12 into the left ventricle 3.

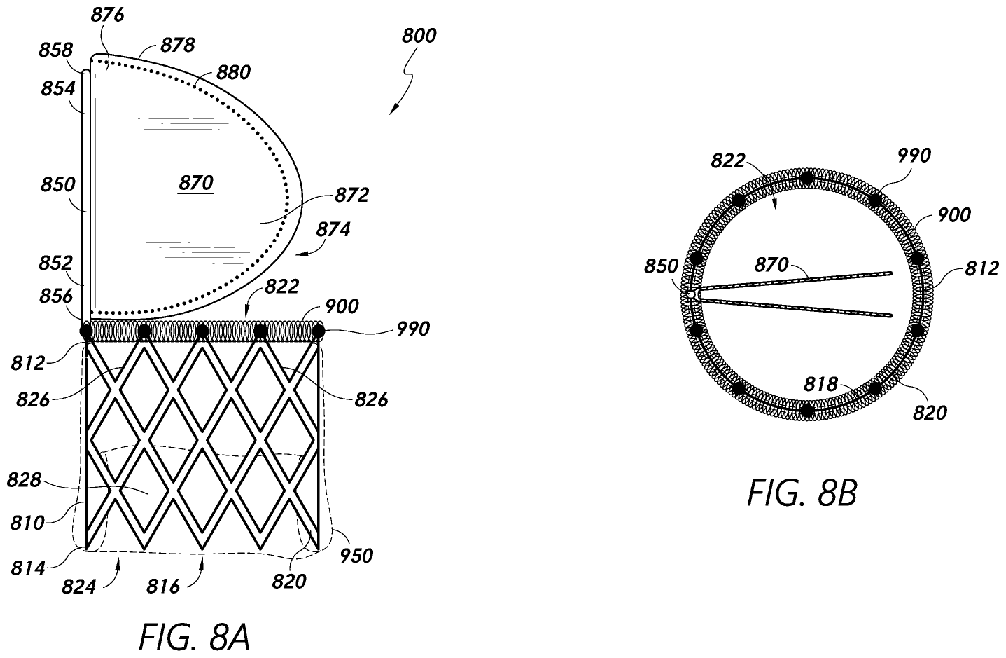

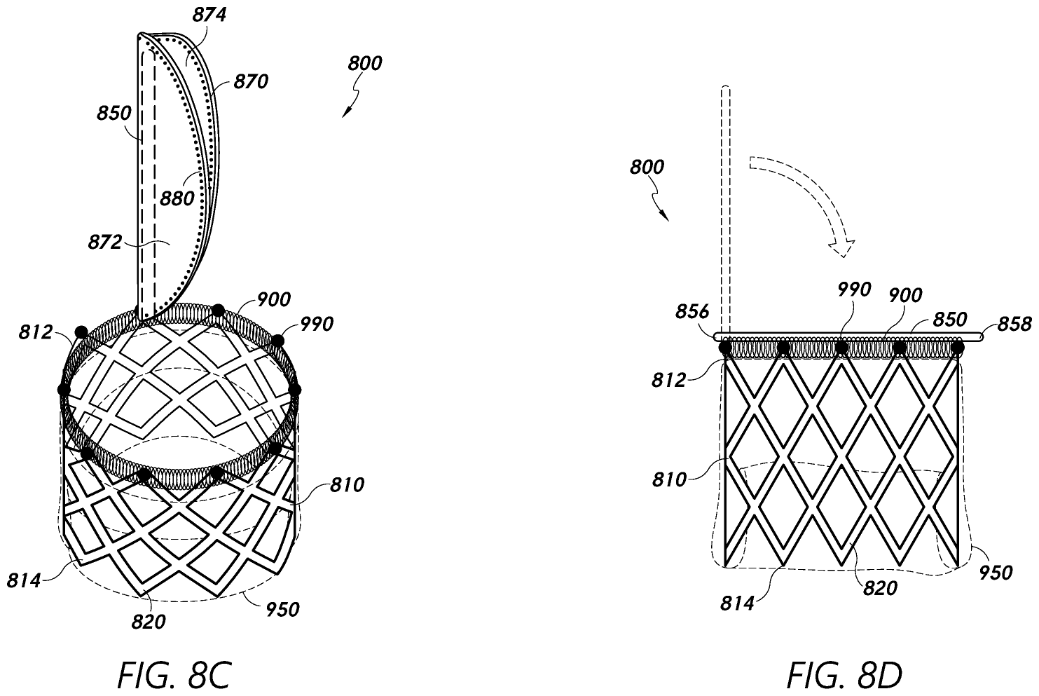

FIGS. 8 through 11 show various views of another example of a prosthetic valve 800. The prosthetic valve 800 can comprise an elongate member 850 coupled to a body member 810, and a covering 870 coupled to the elongate member 850. The can prosthetic valve 800 can comprise a sheath 950 associated with one or more portions of an inner surface 818 and outer surface 820 of the body member 810. A collar 900 and one or more visualization markers 990 can be associated with a first portion 826 of the body member 810, including a first end 812 of the body member 810. FIG. 8A is a side view of the prosthetic valve 800 in an expanded configuration. FIG. 8B is a top-down view, and FIG. 8C is a perspective view, of the prosthetic valve 800 shown in FIG. 8A. FIG. 8D is a side view of the prosthetic valve 800 comprising the elongate member 850 in a diametrical orientation.

The prosthetic valve 800 can comprise one or more features of the prosthetic valve 100 described with reference to FIGS. 2 through 4. Referring to FIG. 8A, for example, the prosthetic valve 800 can comprise a body member 810 with a conduit 816 extending therethrough. The body member 810 can comprise a first end 812 and a second end 814. The body member 810 can comprise an inner surface 818 oriented toward the conduit 816, and an outer surface 820 oriented toward tissue at the target tissue site. The elongate member 850 can comprise a first end portion 852 and a second end portion 854. The elongate member 850 can be associated with the first end 812 of the body member 810. In some embodiments, the elongate member 850 can be integral with the body member 810. In some embodiments, the body member 810 and the elongate member 850 can comprise a shape memory material such that the elongate member 850 can be shape set to move from a longitudinal orientation to a diametrical orientation while the prosthetic valve 100 transforms from a compressed configuration to an expanded configuration. Alternatively, the elongate member 850 can be coupled to the body member 810, such as at the first end portion 852 of the elongate member 850. The covering 870 can be coupled to at least a portion of the length of the elongate member 850. In some embodiments, the covering 870 can be coupled to an entire or substantially an entire length of the elongate member 850. The covering 870 can comprise a unitary covering member, such as a single leaflet. The elongate member 850 can be coupled to the covering 870 along at least a portion of a lateral dimension of the covering 870. As described herein, in some embodiments, the body member 810 can form a cylindrical shape. In some embodiments, the first end 812, first opening 822, the second end 814 and/or the second opening 824 can each comprise a circular or substantially circular shape. The covering 870 can have a shape similar to or the same as that of the first end 812 and/or the first opening 822, such as a circular or substantially circular shape. The covering 870 can be coupled to the elongate member 850 at least partially along a diameter of the covering 870. In some embodiments, the covering 870 can be coupled to the elongate member 850 along the entire or substantially entire diameter of the covering 870.

The covering 870 can be mechanically reinforced at least partially around an edge portion 876. In some embodiments, a reinforcement feature 880 can be associated with at least a portion of the edge portion 876. In FIG. 8A, the elongate member 850 is shown as extending away from the body member 810, for example assuming a longitudinal orientation. The covering 870 is shown as being bent back upon itself along the portion coupled to the elongate member 850 such that the first surface 872 is shown in FIG. 8A. The reinforcement feature 880 can be associated with a first surface 872 of the covering 870, the first surface 872 being configured to be oriented away from the body member 810 while the covering 870 is positioned over the first end 812 of the body member 810. In some embodiments, the reinforcement feature 880 can be continuous, for example circumscribing a portion of the covering 870. In some embodiments, the reinforcement feature 880 can be associated with the entire or substantially entire edge portion 876.

The reinforcement feature 880 can be configured to provide added mechanical strength for the covering 870. The reinforcement feature 880 can facilitate secure positioning of the covering 870 over the first end 812 of the body member 810 while the prosthetic valve 800 is in the closed state. The reinforcement 880 can reduce or prevent deformation of the covering 870, such as due to pressure exerted thereon by fluid pushing against the first surface 872 of the covering 870. The reinforcement feature 880 can prevent the covering 870 from collapsing into the conduit 116 of the body member 810 due to pressure exerted thereon.

In some embodiments, a reinforcement feature can be associated with the first surface 872 of the covering 870, with a second surface 874 of the covering 870 configured to be oriented toward the body member 810 while the covering 870 is positioned over the first end 812 of the body member 810, and/or imbedded within the covering 870. The reinforcement feature can be continuously or discontinuously arranged around the edge portion 876. In some embodiments, the reinforcement feature can comprise an additional thickness of the covering 870. The reinforcement feature can comprise one or more additional layers of the material used for the remainder of the covering 870. For example, the edge portion 876 of the covering 870 can be folded back onto itself such that the reinforcement feature comprises one or more additional layers of the material used for the remaining portions of the covering 870. In some embodiments, the reinforcement feature can comprise one or more additional layers of the material used for the covering 870 bonded to the edge portion 876. In some embodiments, the reinforcement feature can comprise one or more types of materials different from that used for the covering 870.

In some embodiments, the prosthetic valve 800 can comprise a collar 900 associated with a first end portion 826 of the body member 810, including the first end 812 of the body member 810. In some embodiments, the collar 900 can be coupled to and/or integrated with the first end 812. The collar 900 can comprise one or more longitudinal portions 902 which extend along an axis having a same or similar orientation as the longitudinal axis of the body member 110. The longitudinal portion 902 can extend beyond the first end 812. The collar 900 can comprise one or more lateral portions 904 which extend along an axis perpendicular or substantially perpendicular to that of the longitudinal portion 902. For example, the one or more lateral portions 904 can extend along an axis perpendicular or substantially perpendicular to the longitudinal axis of the body member 810. The one or more lateral portions 904 can comprise at least a portion which extends laterally from the outer surface 820 of the body member 810. The collar 900 can be continuously around the entire first end 812 of the body member 810.

In some embodiments, the collar 900 can be configured to reduce or prevent damage of tissue at the target site due to contact between the tissue and the first end portion 826 and/or first end 812 of the body member 810. For example, longitudinal and/or lateral portions 902, 904 can serve as protection against abrasion of the tissue. In some embodiments, the collar 900 can be configured to facilitate secure positioning of the prosthetic valve 800 at the target site and/or improve or eliminate leakage of fluid between the prosthetic valve 800 and the target tissue while the prosthetic valve 800 is at the target site. Lateral portions 904 of the collar 900 can provide added structural features to stably position the prosthetic valve 800 at the target site and/or seal an opening at which the prosthetic valve 800 is positioned.

In some embodiments, the collar 900 can comprise portions looped around the first end 812 of the body member 810. As described herein, the body member 810 can comprise a wired configuration. The collar 900 can comprise portions looped through wired portions at the first end portion 826. Looping the collar 900 around the first end portion 826 can serve as protection against abrasion of surrounding tissue at the target site, facilitate secure positioning of the prosthetic valve 800 and/or improve or eliminate leakage of fluid around the prosthetic valve 800.

In some embodiments, the collar 900 can comprise polyethylene terephthalate (PET). In some embodiments, the collar 900 can comprise a polyethylene terephthalate (PET) material, including fibers, looped around the first end portion 826 of the body member 810. For example, the polyethylene terephthalate (PET) material can be looped around the first end 812 and extend around the entire circumference of the first end 812. Other pliable biocompatible polymeric material may be used in the alternative or in combination.

A collar can be partially or entirely around the first end 812. In some embodiments, a collar can be continuously around the first end 812. In some embodiments, a collar can comprise one or more discrete portions, including discrete portions at regular intervals around the first end 812.

In some embodiments, the prosthetic valve 800 can comprise a one or more visualization markers 990. The one or more visualization markers 990 can facilitate visualization of the position and/or orientation of the prosthetic valve 800, such as during delivery of the prosthetic valve 800 to the target site. For example, the one or more visualization markers 990 can be one or more radiopaque markers. In some embodiments, the visualization markers 990 can be coupled to and/or integrated with the first end portion 826, including the first end 812 of the body member 810. In some embodiments, the visualization markers 990 can be coupled to and/or integrated as a part of the collar 900. The one or more visualization markers 990 can be around first end portion 826, such as at positions around the first end 812, including at regularly spaced positions. For example, the visualization markers 990 can be at one or more positions around a circumference of the collar 900, including at a plurality of regularly spaced positions.

Referring again to FIG. 8A, the prosthetic valve 800 can comprise a sheath 950 associated with the body member 810. The sheath 950 can be associated with at least a portion of the outer surface 820 and/or at least a portion of the inner surface 818 of the body member 810. The sheath 950 can be associated with the entire inner surface 818 and/or outer surface 820 of the body member 810. In some embodiments, the sheath 950 can be associated with a portion of the inner surface 818 and/or a portion of the outer surface 820 of the body member 810. For example, the sheath 950 can be coupled to the inner surface 818 and/or outer surface 820 through any number of methods, including bonding and/or lamination. In some embodiments, the sheath 950 can wrap around the second end 814 of the body member 810 such that the sheath 950 is over both at least a portion of the inner surface 818 and at least a portion of the outer surface 820.

In some embodiments, the sheath 950 can comprise a paravalvular leakage skirt. The sheath 950 can comprise one or more of any number of biocompatible materials configured serve as a shield between the body member 810 and the target tissue. In some embodiments, the sheath 950 can comprise polyethylene terephthalate (PET). The sheath 950 can be configured to reduce or prevent damage to any surrounding tissue at the target site, improve stable positioning of the prosthetic valve 800, and/or diminish or eliminate fluid leakage between the body member 810 and the target tissue while the prosthetic valve 800 is at the target site. The sheath 950 can comprise one or more other biocompatible polymeric material.

In some embodiments, the collar 900 and/or the sheath 950 can comprise one or more of polyethyleneterephthalate (PET), polypropylene (PP), polyethylene (PE), polymethylmethacrylate (PMMA), polystyrene (PS), polyvinylchloride (PVC), polytetrafluoroethylene (PTFE), polyurethane (PU), polyamide (nylon), polyethersulfone (PES), polyetherimide (PEI), polyetheretherketone (PEEK), polyvinylchloride (PVC), and poly(lactic-co-glycolic) acid (PLGA).

FIG. 8B shows a top-down view of the prosthetic valve 800. The collar 900 and the visualization markers 990 are shown. A view of the covering 870 bent back onto itself and the elongate member 850 is shown from the second end portion 854 of the elongate member 850. FIG. 8C shows a perspective view of the prosthetic valve 800 shown in FIG. 8A. The sheath 950 is shown as being associated with both a portion of the inner surface 818 and outer surface 820 of the body member 810. As described herein, the sheath 950 can wrap around the second end 814 of the body member 810 such that the sheath 950 is over both a portion of the inner surface 818 and the outer surface 820.

FIG. 8D shows a side view of the prosthetic valve 800 comprising the elongate member 850 in a diametrical orientation. The elongate member 850 can move from the longitudinal orientation shown in FIG. 8A to the diametrical orientation, such as by pivoting around the first end 856. As described in further detail, the elongate member 850 can be in the diametrical orientation while the prosthetic valve 800 is in the expanded configuration.

In some embodiments, one or both of the first end 812 and the second end 814 of the body member 810 can optionally comprise an atraumatic configuration. The atraumatic configuration can be configured to prevent abrasion and/or damage to surrounding tissue at the target site. For example, the first end 812 can comprise a rounded configuration. As described herein, the body member 810 can comprise a wired configuration. In some embodiments, an atraumatic first end can comprise curved wire portions to form rounded portions at the first end 812. In some embodiments, an atraumatic second end can comprise curved wire portions to form rounded portions at the second end 814.

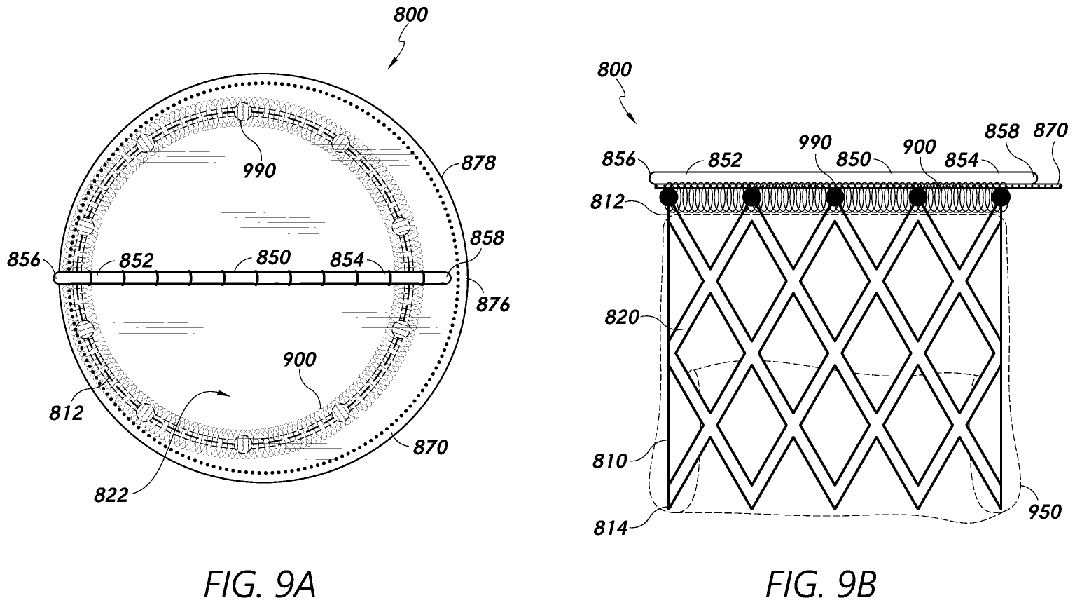

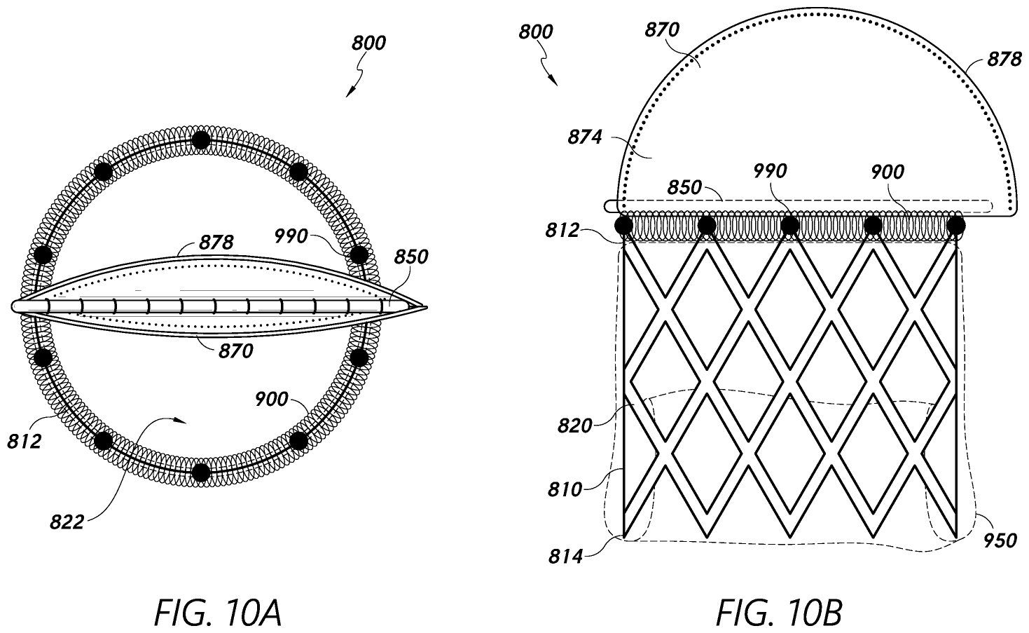

FIG. 9A is a top-down view, and FIG. 9B is a side view, of the prosthetic valve 800 in an expanded configuration and a closed state. FIG. 10A is a top-down view, and FIG. 10B is a side view, of the prosthetic valve 800 in an expanded configuration and an open state. The collar 900 and the visualization markers 990 are shown in the top-down views. The sheath 950 is shown in the side views. Referring to FIGS. 9A and 9B, the covering 870 is shown as being positioned over the first end 812 of the body member 810. The covering 870 is shown as being transparent for illustrative purposes. The covering 870 can comprise a size larger than that of the first end 812, including that of the first opening 822, to facilitate secure positioning of the covering 870 over the first end 812. At least a portion of the edge portion 876 of the covering 870 can extend along a lateral axis beyond the first end 112. The lateral axis can be perpendicular or substantially perpendicular to the longitudinal axis of the body member 810. For example, the covering 870 can comprise a diameter larger than that of the first end 812 such that at least a portion of the edge portion 876 extends laterally beyond the outer surface 820 of the body member 810.

Referring to FIGS. 10A and 10B, a portion of the covering 870 can be positioned away from the first end 812 of the body member 810. The covering 870 can be positioned away from the first end 812 such that fluid can flow through the first opening 822. The covering 870 can be bent back toward itself due to force exerted upon the second surface 874. For example, the second surface 874 is shown in FIG. 10B.

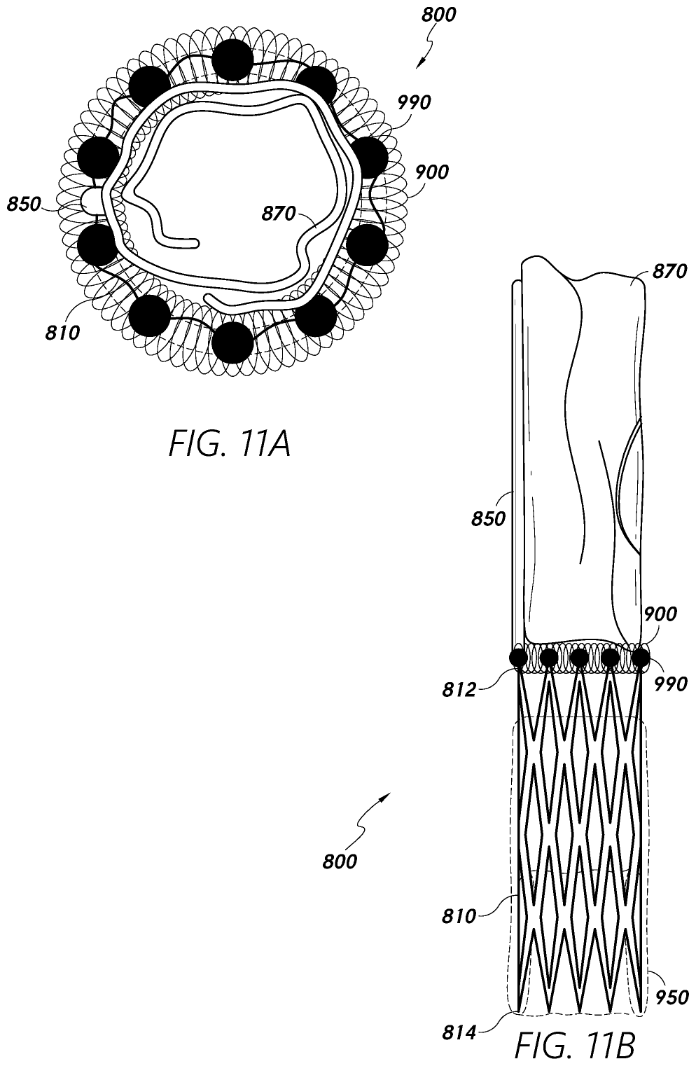

FIG. 11A is a top-down view, and FIG. 11B is a side view, of the prosthetic valve 800 in a compressed configuration. In the compressed configuration, the body member 810 can have a width smaller than that in the expanded configuration. The elongate member 850 is in a longitudinal orientation in the compressed configuration. The elongate member 850 is coming out of the page in FIG. 11A. In FIG. 8B, the elongate member 850 can have a same or similar orientation as that of the body member 810. The covering 870 can be folded so as to assume a narrower configuration. As described herein, the prosthetic valve 800 can be in the compressed configuration while within a delivery catheter during delivery to the target site.

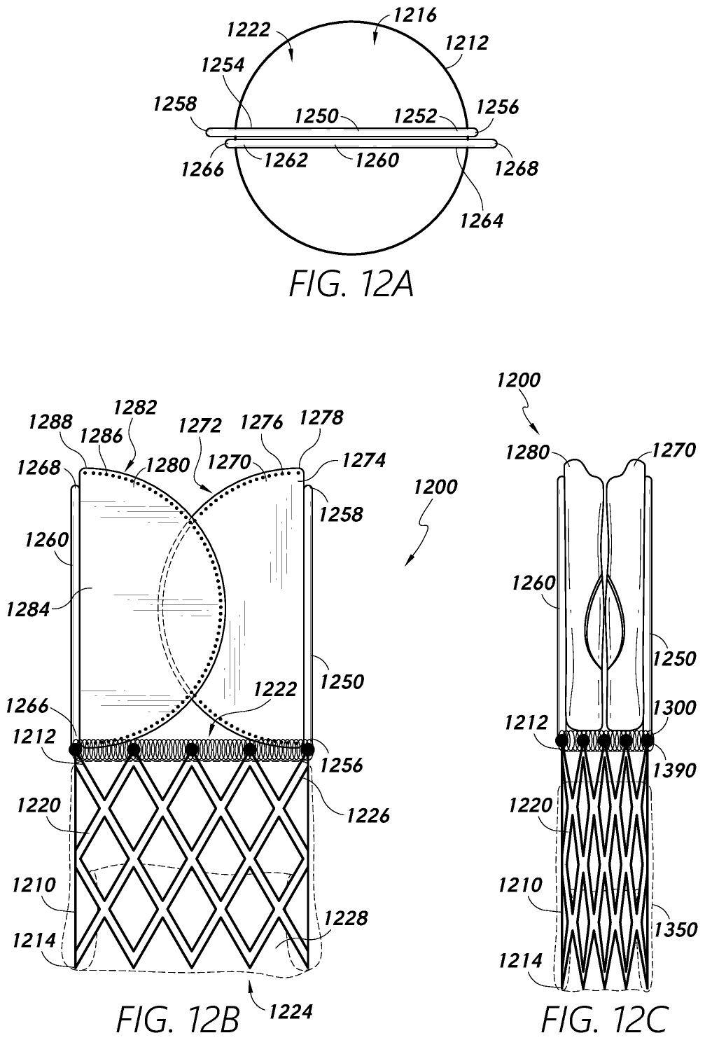

FIGS. 12 through 14 various views of another example of a prosthetic valve 1200. FIG. 12A is a top-down view, and FIGS. 12B and 12C are side views, of the prosthetic valve 1200. The prosthetic valve 1200 can comprise a first elongate member 1250 and a second elongate member 1260 associated with a body member 1210. The body member 1210 can comprise a first end 1212 and a second end 1214. The body member 1210 can comprise a conduit 1216 extending therethrough, for example such that the first end 1212 comprises a first opening 1222 and the second end 1214 comprises a second opening 1224. In some embodiments, the body member 1210 can form a cylindrical shape. For example, the first end 1212 and/or the first opening 1222 can comprise a circular or substantially circular shape. The second end 1214 and/or the second opening 1224 can comprise a circular or substantially circular shape. A first covering 1270 can be coupled to the first elongate member 1250 and a second covering 1280 can be coupled to the second elongate member 1260. The first and second coverings 1270, 1280 are not shown in FIG. 12A for simplicity.

The first elongate member 1250 can comprise a first end portion 1252, including a first end 1256, and a second end portion 1254, including a second end 1258. The second elongate member 1260 can comprise a first end portion 1262, including a first end 1266, and a second end portion 1264, including a second end 1268. In some embodiments, the elongate members 1250, 1260 can be integral with the body member 1210. The elongate members 1250, 1260 can extend from respective positions at the first end 1212 of the body member 1210. In some embodiments, the body member 1210 and the elongate members 1250, 1260 can comprise a shape memory material such that the elongate members 1250, 1260 can be shape set to move from a longitudinal orientation to a diametrical orientation while the prosthetic valve 1200 transforms from a compressed configuration to an expanded configuration. Alternatively, the elongate members 1250, 1260 can be coupled to the body member 1210. In some embodiments, the first and second elongate members 1250, 1260 can be rotatably coupled to respective portions of the body member 1210 at the first end portions 1252, 1262, such as the first end 1256, 1266. For example, the first and second elongate members 1250, 1260 can be coupled to the first end 1212 of the body member 1210 at respective positions.

FIG. 12A shows the first and second elongate members 1250, 1260 in a diametrical orientation. The first and second elongate members 1250, 1260 can be positioned over respective portions of the first end 1212 of the body member 1210. The first and second elongate members 1250, 1260 can extend across respective portions of the first opening 1222. While the first and second elongate members 1250, 1260 are positioned over the first end 1212, no portion of the first elongate member 1250 overlaps with the second elongate member 1260. In some embodiments, the first and second elongate member 1250, 1260 can comprise at least a portion extending along one or more planes perpendicular or substantially perpendicular to a longitudinal axis of the body member 1210. The first and second elongate members 1250, 1260 can be in a side-by-side configuration, including a being parallel or substantially parallel to one another. In some embodiments, the first and second elongate members 1250, 1260 can be adjacent to and in contact with one another. In some embodiments, the first and second elongate members 1250, 1260 can comprise respective portions extending along parallel or substantially parallel portions of the same plane.

FIG. 12B shows the prosthetic valve 1200 in an expanded configuration and FIG. 12C shows the prosthetic valve 1200 in a compressed configuration. In both figures, the elongate members 1250, 1260 are extending away from the body member 1210. The first and second elongate members 1250, 1260 can be in a longitudinal orientation in the configurations shown in FIGS. 12B and 12C. The orientation of the first and second elongate members 1250, 1260 can be similar to or the same as the longitudinal axis of the body member 1210. For example, each of the first and second elongate members 1250, 1260 can have a longitudinal axis parallel or substantially parallel to that of the body member 1210. In some embodiments, the first and second elongate members 1250, 1260 can have the same or similar lengths.

In the expanded configuration shown in FIG. 12B, the first covering 1270 can be coupled to the first elongate member 1250 along at least a portion of the length of the first elongate member 1250. The second covering 1280 can be coupled to the second elongate member 1260 along at least a portion of the length of the second elongate member 1260. The first and second coverings 1270, 1280 can be in an unfolded configuration. The first and second coverings 1270, 1280 can be coupled to the first and second elongate members 1250, 1260 along a respective part of an edge portion 1276, 1286. As described herein, in some embodiments, the first end 1212 and/or the first opening 1222 can have a circular or substantially circular shape. The first and second coverings 1270, 1280 can each comprise a segment of a circle. In some embodiments, the first and second coverings 1270, 1280 can each comprise a semi-circle shape. Together, the first and second coverings 1270, 1280 can close off the first opening 1222 while positioned over the first end 1212. For example, each of the first and second coverings 1270, 1280 can be coupled to the first and second elongate members 1250, 1260 along a linear portion of an outer edge 1278, 1288, respectively. While positioned over the first end 1212, the curved portion of the outer edges 1278, 1288 can be oriented toward the outer surface 1220 of the body member 1210. In some embodiments, the first and second coverings 1270, 1280 can each comprise a semi-circular leaflet. While the leaflets are positioned over the first end 1212 of the body member 1210, the linear portion of the outer edge of each leaflet can be oriented toward that of the other, and the curved portion of the outer edge of each leaflet can be oriented toward a respective portion of the first end 1212.

FIG. 12C shows the prosthetic valve in a compressed configuration. In the compressed configuration, the first and second coverings 1270, 1280 can each be folded. For example, the first and second coverings 1270, 1280 can each comprise a rolled configuration while in the folded configuration. In some embodiments, the rolled configurations can each comprise a longitudinal axis parallel or substantially parallel to that of the body member 1210.

The prosthetic valve 1200 can have one or more other features of the prosthetic valve 800 described with reference to FIGS. 8 through 11. For example, the prosthetic valve 1200 can comprise a sheath 1350 associated with one or more portions of the inner surface 1218 and outer surface 1220 of the body member 1210. A collar 1300 and one or more visualization markers 1390 can be associated with a first portion 1226 of the body member 1210, including a first end 1212 of the body member 1210.

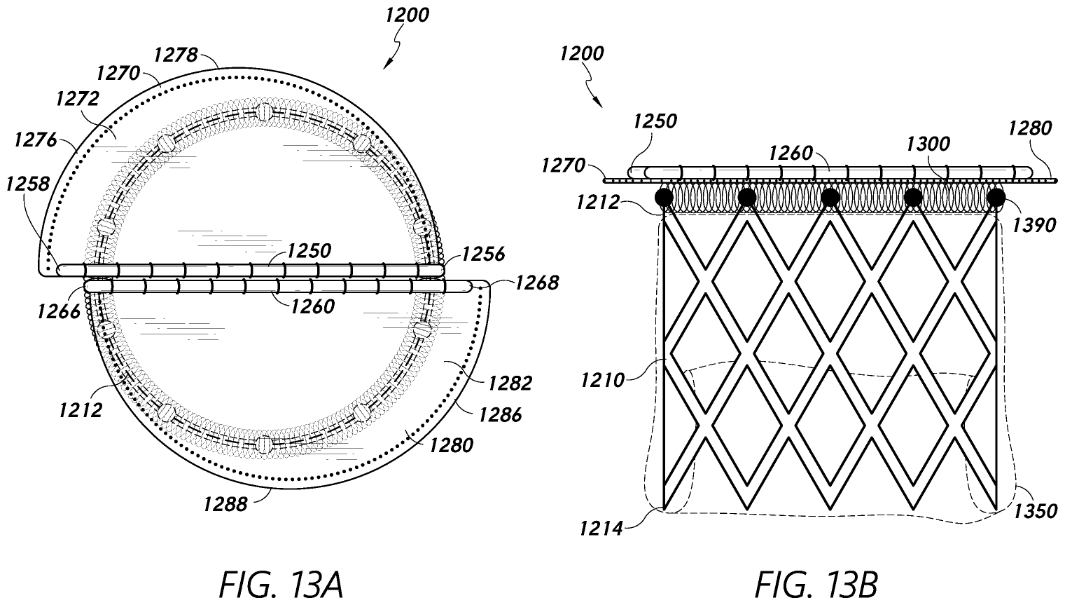

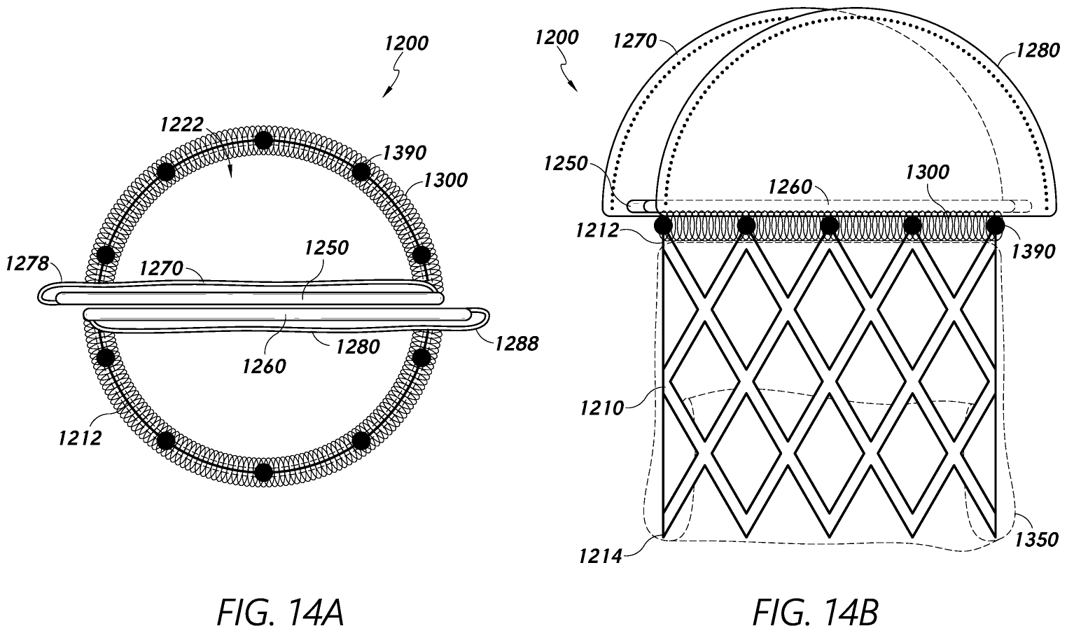

FIG. 13A is a top-down view, and FIG. 13B is a side view, of the prosthetic valve 1200 in a closed state. FIG. 14A is a top-down view, and FIG. 14B is a side view, of the prosthetic valve 1200 in an open state. When the prosthetic valve 1200 is in the expanded configuration, the prosthetic valve 1200 can be configured to alternate between the open state and the closed state. The coverings 1270, 1280 are shown in FIG. 13A as being transparent for illustrative purposes. In the closed state, the first and second coverings 1270, 1280 are shown as being positioned over respective portions of the first end 1212 of the body member 1210. Together, the first and second coverings 1270, 1280, can close off the first opening 1222 at the first end 1212 of the body member 1210 and prevent fluid flow therethrough. Each of the first and second coverings 1270, 1280 can comprise a portion which extends laterally beyond the outer surface 1220 of the body member 1210 at the first end 1212 to facilitate secure positioning of the coverings 1270, 1280 over the first end 1212. The first and second coverings 1270, 1280 can comprise respective portions which extend laterally along a plane perpendicular or substantially perpendicular to the longitudinal axis of the body member 1210. At least a part of an edge portion 1276, 1286 of the coverings 1270, 1280 can be laterally beyond the first end 1212. As described herein, the body member 1210 can form a cylindrical shape. The first end 1212 and the first opening 1222 can each comprise a circular shape. While positioned over the first end 1212, the curved portion of the outer edges 1278, 1288 can be oriented toward the outer surface 1220 of the body member 1210, and the linear portion of the outer edge 1278, 1288 of each covering 1270, 1280 can be oriented toward that of the other.

Referring to FIGS. 14A and 14B, a portion of each of the first and second coverings 1270, 1280 can be positioned away from the first end 1212 of the body member 1210. A portion of the coverings 1270, 1280 can be spaced away from the first end 1212 such that fluid can flow through the first opening 1222. The coverings 1270, 1280 can be bent back and away from the body member 1210 due to force exerted upon the second surfaces 1274, 1284 by fluid flowing from through the body member 1210 the second end 1214 to the first end 1212. The second surfaces 1274, 1284 are shown in FIG. 14B.

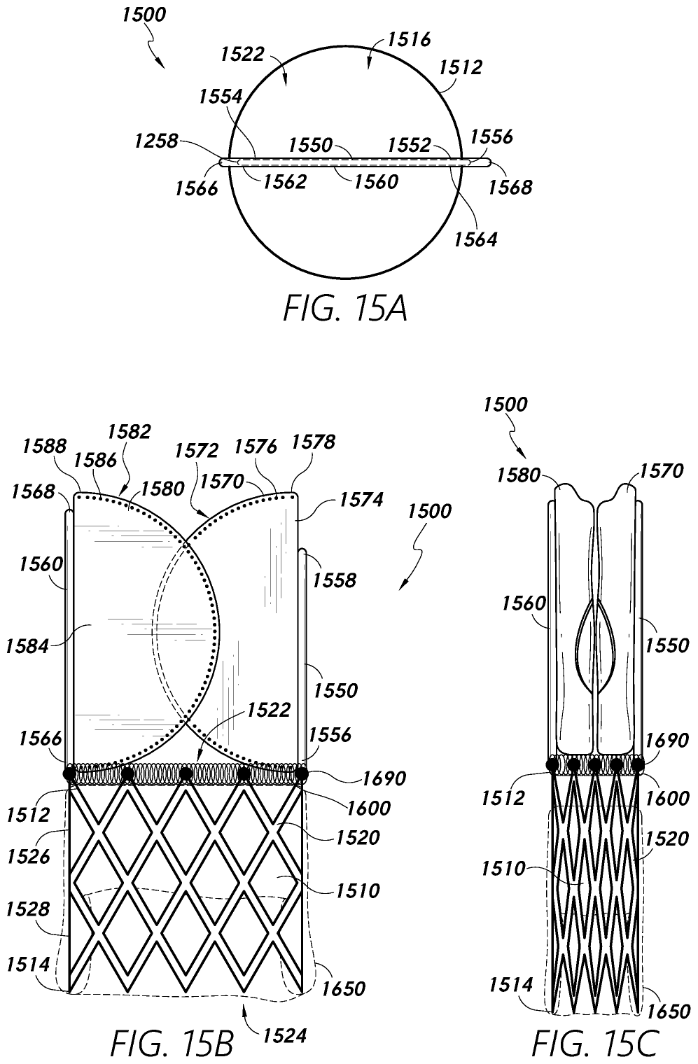

FIGS. 15 through 17 show another example of a prosthetic valve 1500 comprising two elongate members. FIG. 15A is a top-down view, and FIGS. 15B and 15C are side views, of the prosthetic valve 1500. The prosthetic valve 1500 can comprise a first elongate member 1550 and a second elongate member 1560 associated with a body member 1510. The body member 1510 can comprise a first end 1512 and a second end 1514. The body member 1510 can comprise a conduit 1516 extending therethrough, for example such that the first end 1512 comprises a first opening 1522 and the second end 1514 comprises a second opening 1524. FIG. 15A shows the first and second elongate members 1550, 1560 in a diametrical orientation. The first and second elongate members 1550, 1560 can be positioned over the first end 1512 of the body member 1510 one over the other. The first and second elongate members 1550, 1560 can be in a stacked configuration while positioned across the first end 1512 of the body member 1510. For example, in FIG. 15A, the first elongate member 1550 is shown as being positioned below the second elongate member 1560. In some embodiments, corresponding portion of each of the first and second elongate members 1550, 1560 can share a plane parallel or substantially parallel with and/or contains a longitudinal axis of the body member 1510.

The first elongate member 1550 can comprise a first end portion 1552, including a first end 1556, and a second end portion 1554, including a second end 1558. The second elongate member 1560 can comprise a first end portion 1562, including a first end 1566, and a second end portion 1564, including a second end 1568. In some embodiments, the elongate members 1550, 1560 can be integral with the body member 1510, the elongate members 1550, 1560 extending from respective positions at the first end 1512 of the body member 1510. In some embodiments, the body member 1510 and the elongate members 1550, 1560 can comprise a shape memory material such that the elongate members 1550, 1560 can be shape set to move from a longitudinal orientation to a diametrical orientation while the prosthetic valve 1500 transforms from a compressed configuration to an expanded configuration. Alternatively, the elongate members 1550, 1560 can be coupled to the body member 1510. The first and second elongate members 1550, 1560 can be rotatably coupled to respective portions of the body member 1510 at the first end portions 1552, 1562, such as the first end 1556, 1566. For example, the first and second elongate members 1550, 1560 can be coupled to the first end 1512 of the body member 1510 at respective positions.

In some embodiments, the body member 1510 can form a cylindrical shape. For example, the first end 1512 and/or the first opening 1522 can comprise a circular or substantially circular shape. The second end 1514 and/or the second opening 1524 can comprise a circular or substantially circular shape. In some embodiments, the first and second elongate member 1550, 1560 can extend across a diameter of the body member 1510. The first ends 1556, 1566 of the first and second elongate members 1550, 1560 can be at opposing portions of the body member 1510. For example, the first ends 1556, 1566 can be associated with, such as extending from or rotatably coupled to, the body member 1510 at diametrically opposing positions on the first end 1512. A first covering 1570 can be coupled to the first elongate member 1550 and a second covering 1580 can be coupled to the second elongate member 1560. The first and second coverings 1570, 1580 are not shown in FIG. 15A for simplicity.

FIG. 15B shows the prosthetic valve 1500 in an expanded configuration and FIG. 15C shows the prosthetic valve 1200 in a compressed configuration. In both figures, the elongate members 1550, 1560 are extending away from the body member 1510. The first and second elongate members 1550, 1560 can be in a longitudinal orientation in the configurations shown in FIGS. 15B and 15C. The orientation of the first and second elongate members 1550, 1560 can be similar to or the same as the longitudinal axis of the body member 1510. For example, each of the first and second elongate members 1550, 1560 can have a longitudinal axis parallel or substantially parallel to that of the body member 1510. The first elongate member 1550 can be configured to pivot around its coupling with the body member 1510 such that it is positioned over the first end 1512 before the second elongate member 1560 is positioned over the first end 1512 such that the first elongate member 1550 can be below the second elongate member 1560. In some embodiments, the first elongate member 1550 can have a longitudinal dimension shorter than that of the second elongate member 1560. The first elongate member 1550 can be shorter than the second elongate member 1560 such that the first and second elongate members 1550, 1560 can be in a stacked configuration when the prosthetic valve 1500 is in a closed state, where the first elongate member 1550 is positioned below the second elongate member 1560.

In the expanded configuration shown in FIG. 15B, the first covering 1570 can be coupled to the first elongate member 1550 along at least a portion of the length of the first elongate member 1550. The second covering 1580 can be coupled to the second elongate member 1560 along at least a portion of the length of the second elongate member 1560. The first and second coverings 1570, 1580 can be in an unfolded configuration. The first and second coverings 1570, 1580 can be coupled to the first and second elongate members 1550, 1560 along a respective part of an edge portion 1576, 1578. As described herein, in some embodiments, the first end 1512 and/or the first opening 1522 can have a circular or substantially circular shape. The first and second coverings 1570, 1580 can each comprise a segment of a circle. In some embodiments, the first and second coverings 1570, 1580 can each comprise a semi-circle shape. Together, the first and second coverings 1570, 1580 can close off the first opening 1522 while positioned over the first end 1512. For example, each of the first and second coverings 1570, 1580 can be coupled to the first and second elongate members 1550, 1560 along a linear portion of an outer edge 1578, 1588, respectively. While positioned over the first end 1512, the curved portion of the outer edges 1578, 1588 can be oriented toward the outer surface 1520 of the body member 1510. In some embodiments, the first and second coverings 1570, 1580 can each comprise a semi-circular leaflet. While the leaflets are positioned over the first end 1512 of the body member 1510, the linear portion of the outer edge of each leaflet can be oriented toward that of the other, and the curved portion of the outer edge of each leaflet can be oriented toward a respective portion of the first end 1512.

FIG. 15C shows the prosthetic valve in a compressed configuration. In the compressed configuration, the first and second coverings 1570, 1580 can each be folded, for example assuming a rolled configuration. In some embodiments, the rolled configurations can each comprise a longitudinal axis parallel or substantially parallel to that of the body member 1510.

The prosthetic valve 1500 can have one or more other features of the prosthetic valve 800 described with reference to FIGS. 8 through 11. For example, the prosthetic valve 1500 can comprise a sheath 1650 associated with one or more portions of the inner surface 1518 and outer surface 1520 of the body member 1510. A collar 1600 and one or more visualization markers 1690 can be associated with a first portion 1526 of the body member 1510, including a first end 1512 of the body member 1510.

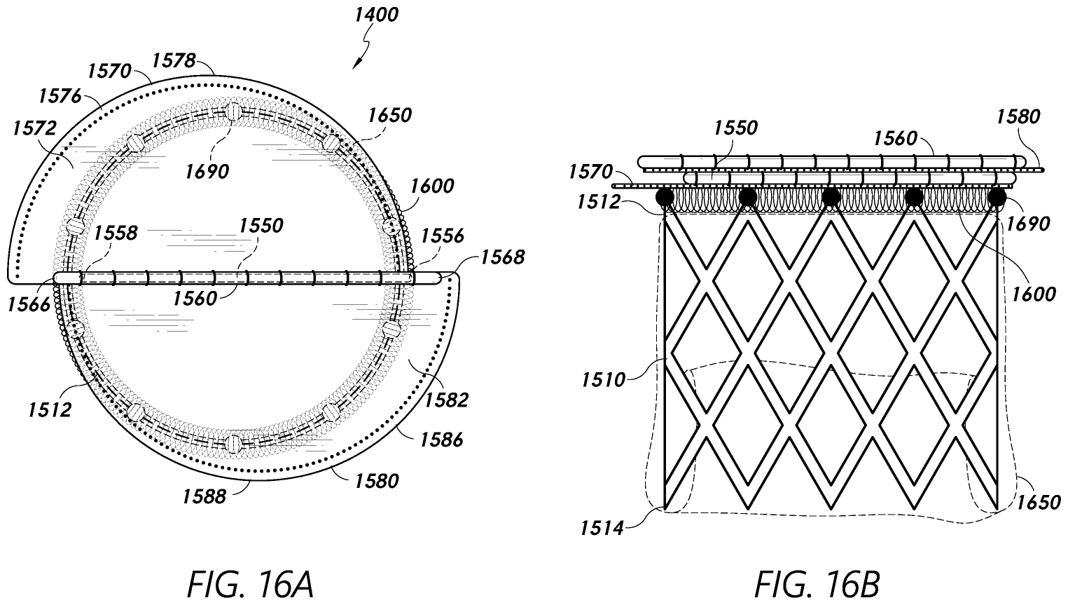

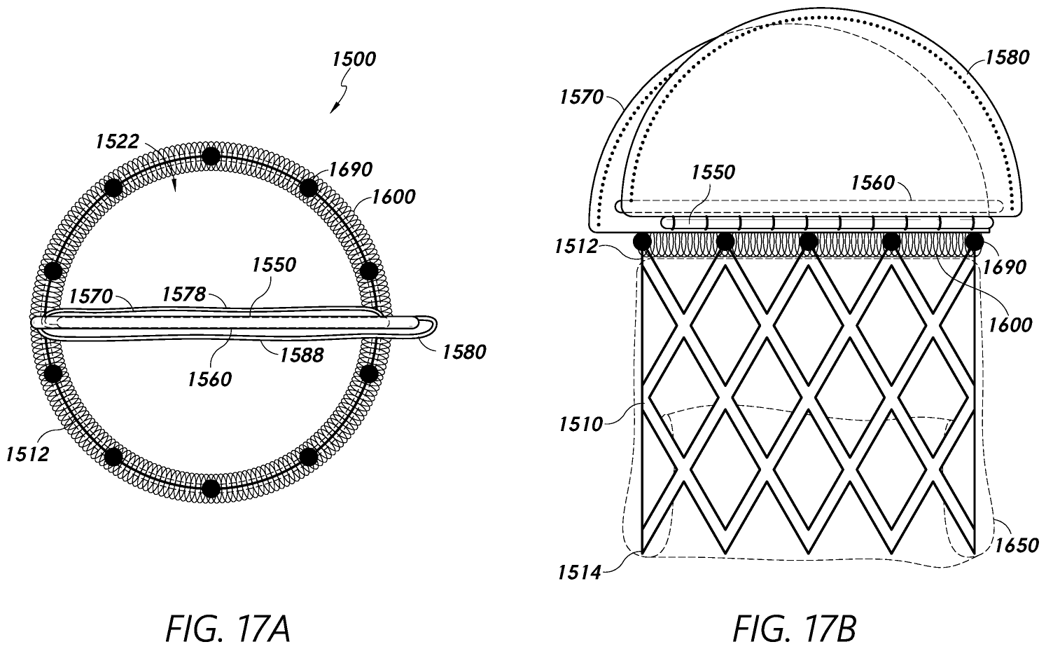

FIG. 16A is a top-down view, and FIG. 16B is a side view, of the prosthetic valve 1500 in a closed state. FIG. 17A is a top-down view, and FIG. 17B is a side view, of the prosthetic valve 1500 in an open state. When the prosthetic valve 1500 is in the expanded configuration, the prosthetic valve 1500 can be configured to alternate between the open state and the closed state. The first and second coverings 1570, 1580 are shown as being transparent in FIG. 16A for illustrative purposes. In the closed state, the first and second coverings 1570, 1580 are shown as being positioned over respective portions of the first end 1512 of the body member 1510 such that the first and second coverings 1570, 1580 together can close off the first opening 1222. Each of the first and second coverings 1570, 1580 can comprise a portion which extends laterally beyond the outer surface 1520 of the body member 1510 at the first end 1512 to facilitate secure positioning of the coverings 1570, 1580 over the first end 1512. The first and second coverings 1570, 1580 can comprise respective portions which extend laterally along a plane perpendicular or substantially perpendicular to the longitudinal axis of the body member 1510. In some embodiments, while positioned over the first end 1512, the curved portion of the outer edges 1578, 1588 can be oriented toward the outer surface 1520 of the body member 1510, and the linear portion of the outer edge 1578, 1588 of each covering 1570, 1580 can be oriented toward that of the other.

Referring to FIGS. 17A and 17B, a portion of each of the first and second coverings 1570, 1580 can be positioned away and spaced from the first end 1512 of the body member 1510, such that fluid can flow through the first opening 1522. The coverings 1570, 1580 can both be bent back and away from the body member 1510 due to force exerted upon the second surfaces 1574, 1584 by fluid flowing from through the body member 1510 the second end 1514 to the first end 1512. The second surfaces 1574, 1584 are shown in FIG. 17B.

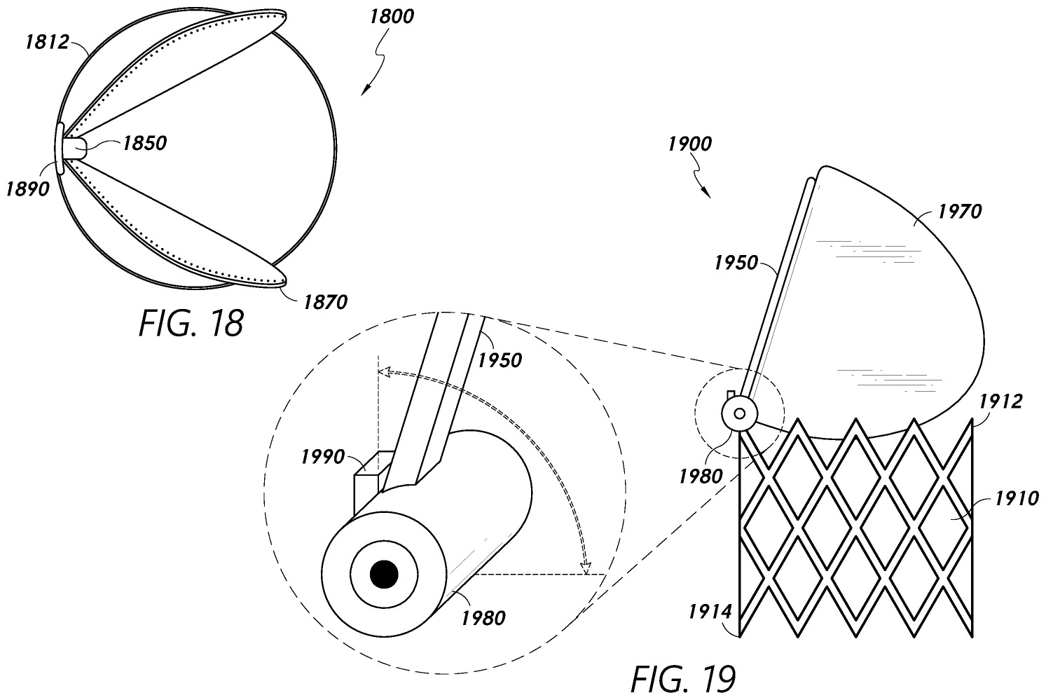

FIG. 18 is a top-down view of an example of a prosthetic valve 1800 comprising an elongate member 1850 coupled to a body member 1810 via a hinge connector 1890. A covering 1870 can be coupled to the elongate member 1850. The elongate member 1850 can comprise a first end portion oriented toward the hinge connector 1890 and a second end portion oriented away from the hinge connector 1890. The hinge connector 1890 can couple the elongate member 1850 to the first end 1812 of the body member 1810, such as at the first end portion of the elongate member 1850. The hinge connector 1890 can enable rotation of the elongate member 1850 around its first end relative to the body member 1810 such that the elongate member 1850 can move between a longitudinal orientation and a diametrical orientation. The elongate member 1850 can be in the longitudinal orientation to facilitate delivery of the prosthetic valve 1800 to the target site. The elongate member 1850 can be in the diametrical orientation while at the target site. The hinge connector 1890 can enable rotation of the elongate member 1850 around its first end to move the elongate member 1850 from the longitudinal orientation to the diametrical orientation.

The prosthetic valve 1800 can have one or more other features of the prosthetic valve 800 described with reference to FIGS. 8 through 11. For example, the prosthetic valve 1900 can comprise a sheath associated with one or more portions of the inner surface and outer surface of the body member 1810. A collar and one or more visualization markers can be associated with the body member 1810.

FIG. 19 shows an example of a prosthetic valve 1900 comprising a hinge connector 1980 and a rotation limiter 1990 configured to limit the rotation of the hinge connector 1980. The prosthetic valve 1900 can comprise an elongate member 1950 coupled to a body member 1910, and a covering 1970 coupled to the elongate member 1950. The hinge connector 1980 can be configured to couple the elongate member 1950 to the first end 1912 of the body member 1910, such as the first end portion 1952 of the elongate member 1950 to the body member 1910. In some embodiments, the hinge connector 1980 can be configured to couple the first end 1956 of the elongate member 1950 to the first end of the body member 1910. The rotation limiter 1990 can be configured to restrict the degree of rotation of the hinge connector 1980, thereby limiting the rotation of the elongate member 1950 relative to the body member 1910.

In some embodiments, the rotation limiter 1990 can be associated with the hinge connector 1980. For example, the rotation limiter 1990 can be integral with the hinge connector 1980. The rotation limiter 1990 can comprise a protrusion configured to contact the elongate member 1950 and/or a rotatable portion of the hinge connector 1980 to stop further rotation of the elongate member 1950 and/or the rotatable portion of the hinge connector 1980.

The prosthetic valve 1900 can have one or more other features of the prosthetic valve 1800 described with reference to FIG. 18. In some embodiments, the prosthetic valve 1900 can comprise a sheath associated with one or more portions of the inner surface and outer surface of the body member 1910. A collar and one or more visualization markers can be associated with the body member 1910.

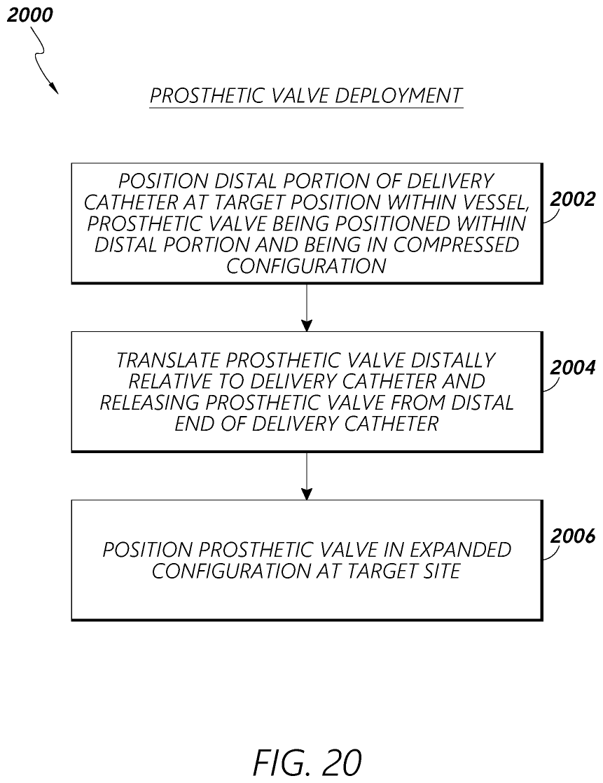

FIG. 20 is a flow diagram of an example of a deployment process 2000 for deploying a prosthetic valve. The prosthetic valve can comprise one or more features described herein. For example, the prosthetic valve can be prosthetic valve 100, prosthetic valve 800, prosthetic valve 1200, prosthetic valve 1500, prosthetic valve 1800 or prosthetic valve 1900, as described herein. In block 2002, the deployment process 2000 can involve positioning a distal portion of a delivery catheter at a target position within a vessel, valve, chamber and/or lumen. In some embodiments, the prosthetic valve can comprise a body member comprising a first end and a second end, an elongate member comprising a first end coupled to the first end of the body member, and a covering coupled to the elongate member along a longitudinal axis of the elongate member. The prosthetic valve can be in a compressed configuration while positioned within the distal portion of the delivery catheter. A longitudinal axis of the elongate member, when the prosthetic valve is in the compressed configuration, can be parallel to a longitudinal axis of the body member. The covering can be in a folded configuration while the prosthetic valve is in the compressed configuration.

In block 2004, the deployment process 2000 can involve translating the prosthetic valve distally relative to the delivery catheter and releasing the prosthetic valve from a distal end of the delivery catheter. In some embodiments, translating the prosthetic valve can comprise contacting the prosthetic valve with a pusher shaft and pushing the prosthetic valve distally with the pusher shaft. The prosthetic valve can assume an expanded configuration after being released from the distal end of the delivery catheter. For example, the body member and covering can each comprise shape memory material such that after being released from the constraint of the delivery catheter, the body member and the covering can expand to assume a respective expanded configuration.

In block 2006, the deployment process 2000 can involve positioning the prosthetic valve in the expanded configuration at a target site. Positioning the prosthetic valve in the expanded configuration at the target site can comprise positioning the prosthetic valve in the heart.

In some embodiments, a process for delivering the prosthetic valve to the target site can comprise a minimally invasive transcatheter delivering technique. For example, the prosthetic valve can be delivered to a target site via the superior vena cava (SVC) or via the inferior vena cava (IVC). In some embodiments, a transjugular or trans-subclavian approach can be used. Alternatively, a transfemoral approach can be used to position the delivery system into the inferior vena cava.

Releasing the prosthetic valve from the distal end of the delivery catheter can comprise releasing the prosthetic valve comprising the first end of the body member oriented proximally and the second end of the body member oriented distally.

In some embodiments, the distal portion of the delivery catheter can be advanced into a right atrium. The distal portion of the delivery catheter can be advanced through the septum from the right atrium into the left atrium. The distal portion of the delivery catheter can be positioned at a target position in the left atrium adjacent to an opening in the left atrium to a pulmonary vein. Releasing the prosthetic valve can comprise positioning at least a portion of the body member within the pulmonary vein. While positioned at the target site, and in an open state, the prosthetic valve can be configured to permit blood flow from the pulmonary vein into the left atrium. In a closed state, the prosthetic valve can be configured to prevent blood flow from the left atrium into the pulmonary vein.

In some embodiments, the distal portion of the delivery catheter can be advanced into a right atrium. The distal portion of the delivery catheter can be positioned adjacent to a coronary sinus ostium in the right atrium. Releasing the prosthetic valve can comprise positioning at least a portion of the body member within the coronary sinus. In an open state, the prosthetic valve can be configured to permit blood flow from the coronary sinus into the right atrium. In a closed state, the prosthetic valve can be configured to prevent blood flow from the right atrium into the coronary sinus.

In some embodiments, the distal portion of the delivery catheter can be advanced into a right atrium and from the right atrium into the right ventricle through the tricuspid valve. The distal portion of the delivery catheter can be advanced through the septum from the right ventricle into the left ventricle. The distal portion of the delivery catheter can be positioned at a target position in the left ventricle adjacent to a mitral valve. Releasing the prosthetic valve can comprise positioning at least a portion of the body member within the mitral valve. In an open state, the prosthetic valve can be configured to permit blood flow from the left atrium into the left ventricle. In a closed state, the prosthetic valve can be configured to prevent blood flow from the left ventricle into the left atrium.

Releasing the prosthetic valve from the distal end of the delivery catheter can comprise releasing the prosthetic valve comprising the first end of the body member oriented distally and the second end of the body member oriented proximally.

In some embodiments, the distal portion of the delivery catheter can be advanced into a right atrium, and through the tricuspid valve from the right atrium into the right ventricle. The distal portion of the delivery catheter can be advanced through the septum from the right ventricle into the left ventricle. The distal portion of the delivery catheter can be positioned at a target position in the left ventricle adjacent to an opening in the left ventricle to the aorta. Releasing the prosthetic valve can comprise positioning at least a portion of the body member within an aortic valve. While positioned at the target site and in an open state, the prosthetic valve can be configured to permit blood flow from the left ventricle into the aorta. In a closed state, the prosthetic valve can be configured to prevent blood flow from the aorta into the left ventricle.

In some embodiments, the distal portion of the delivery catheter can be advanced into a right atrium. The distal portion of the delivery catheter can be positioned adjacent to a tricuspid valve. Releasing the prosthetic valve can comprise positioning at least a portion of the body member within the tricuspid valve. While positioned at the target site and in an open state, the prosthetic valve can be configured to permit blood flow from the right atrium into the right ventricle. In a closed state, the prosthetic valve can be configured to prevent blood flow from the right ventricle into the right atrium.

In some embodiments, the distal portion of the delivery catheter can be advanced into a right atrium. The distal portion of the delivery catheter can be advanced from the right atrium into the left atrium through the septum. The distal portion of the delivery catheter can be positioned at a target position adjacent to a mitral valve. Releasing the prosthetic valve can comprise positioning at least a portion of the body member within the mitral valve. While positioned at the target site and in an open state, the prosthetic valve can be configured to permit blood flow from the left atrium into the left ventricle. In a closed state, the prosthetic valve can be configured to prevent blood flow from the left ventricle into the left atrium.

In some embodiments, the distal portion of the delivery catheter can be advanced into an inferior vena cava. The distal portion of the delivery catheter can be positioned adjacent to an opening of the inferior vena cava into a right atrium. Releasing the prosthetic valve can comprise positioning at least a portion of the body member within the inferior vena cava. In an open state, the prosthetic valve can be configured to permit blood flow from the inferior vena cava into the right atrium. In a closed state, the prosthetic valve can be configured to prevent blood flow from the right atrium into the inferior vena cava.

In some embodiments, the distal portion of the delivery catheter can be advanced into a superior vena cava. The distal portion of the delivery catheter can be positioned to be adjacent to an opening of the superior vena cava into a right atrium. Releasing the prosthetic valve can comprise positioning at least a portion of the body member within the superior vena cava. In an open state, the prosthetic valve can be configured to permit blood flow from the superior vena cava into the right atrium. In a closed state, the prosthetic valve can be configured to prevent blood flow from the right atrium into the superior vena cava.

### Additional Embodiments

Depending on the embodiment, certain acts, events, or functions of any of the processes or algorithms described herein can be performed in a different sequence, may be added, merged, or left out altogether. Thus, in certain embodiments, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain embodiments require at least one of X, at least one of Y and at least one of Z to each be present.

It should be appreciated that in the above description of embodiments, various features are sometimes grouped together in a single embodiment, Figure, or description thereof for the purpose of streamlining the disclosure and aiding in the understanding of one or more of the various inventive aspects. This method of disclosure, however, is not to be interpreted as reflecting an intention that any claim requires more features than are expressly recited in that claim. Moreover, any components, features, or steps illustrated and/or described in a particular embodiment herein can be applied to or used with any other embodiment(s). Further, no component, feature, step, or group of components, features, or steps are necessary or indispensable for each embodiment. Thus, it is intended that the scope of the inventions herein disclosed and claimed below should not be limited by the particular embodiments described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example embodiments belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

What is claimed is:

1. A prosthetic valve comprising:
a body member comprising a first end and a second end, the body member defining a conduit and being configured to assume a compressed configuration and an expanded configuration, a lateral dimension of the body member being larger in the expanded configuration than that in the compressed configuration;
an elongate member having a first end associated with the first end of the body member, the elongate member being configured to move between a longitudinal orientation and a diametrical orientation; and
a covering coupled to the elongate member and configured to cover at least a portion of the conduit at the first end of the body member when the elongate member is in the diametrical orientation.

2. The prosthetic valve of claim 1, wherein:
when the prosthetic valve is in the expanded configuration, the prosthetic valve is configured to alternate between an open state and a closed state,
when the prosthetic valve is in the open state, at least a portion of the covering is configured to be spaced from the first end of the body member to open the first end to fluid flow, and
when the prosthetic valve is in the closed state, the covering is configured to close off the first end of the body member from fluid flow.

3. The prosthetic valve of claim 1, wherein, when the prosthetic valve is in the expanded configuration, the body member forms a cylindrical shape and the elongate member is configured to be positioned across a diameter of the first end of the body member, and the first and second ends of the elongate member are configured to be coupled to diametrically opposing positions on the first end of the body member.

4. The prosthetic valve of claim 1, wherein the elongate member is rotatably coupled to the first end of the body member.

5. The prosthetic valve of claim 4, wherein the prosthetic valve comprises a hinge connector rotatably coupling the first end of the elongate member and the first end of the body member.

6. The prosthetic valve of claim 1, wherein the elongate member is integral with the body member and the elongate member and the body member comprises a shape memory material shape set to position the elongate member across the first end of the body member when the prosthetic valve is in the expanded configuration.

7. The prosthetic valve of claim 1, wherein, when the prosthetic valve is in the expanded configuration, a second end of the elongate member is coupled to the first end of the body member.

8. The prosthetic valve of claim 1, wherein the covering is in a rolled configuration while the prosthetic valve is in the compressed configuration, the rolled configuration comprising a longitudinal axis parallel to that of the body member.

9. The prosthetic valve of claim 1, wherein:
the covering is one leaflet, the one leaflet being coupled to the elongate member along a longitudinal dimension of the elongate member, and
when the prosthetic valve is in the expanded configuration and the closed state, the one leaflet is configured to be positioned over the first end of the body member to close off the fluid flow.

10. The prosthetic valve of claim 1, wherein:
the covering comprises a first leaflet and a second leaflet, each of the first and second leaflets being coupled to the elongate member along a longitudinal dimension of the elongate member, and
when the prosthetic valve is in the expanded configuration and the closed state, the first and second leaflets are each configured to cover a corresponding portion of the first end of the body member to close off the fluid flow.

11. The prosthetic valve of claim 10, wherein:
the first and second leaflets are each rotatably coupled to the elongate member, and
when the prosthetic valve is in the open state, the first and second leaflets are configured to be pivotally rotated around the elongate member and away from the first end of the body member.

12. The prosthetic valve of claim 1, wherein the first end of the body member comprises an atraumatic configuration.

13. A prosthetic valve comprising:
a body member comprising a first end and a second end, the body member defining a conduit extending therethrough and being configured to assume a compressed configuration and an expanded configuration, a lateral dimension of the body member being larger in the expanded configuration than that in the compressed configuration;
a first elongate member comprising a first end and a second end, the first end of the first elongate member being associated with the first end of the body member at a first position on the first end of the body member;
a second elongate member comprising a first end and a second end, the first end of the second elongate member being associated with the first end of the body member at a second position on the first end of the body member, the first and second elongate members being configured to move between a longitudinal orientation and a diametrical orientation; and
a first covering coupled to the first elongate member and a second covering coupled to the second elongate member, the first and second coverings being configured to at least partially cover respective portions of the conduit at the first end of the body member when the first and second elongate members are in the diametrical orientation.

14. The prosthetic valve of claim 13, wherein:
when the prosthetic valve is in the expanded configuration, the prosthetic valve is configured to alternate between an open state and a closed state,
when the prosthetic valve is in the open state, at least a portion of the first and second coverings are configured to be spaced from the first end of the body member to open the first end to fluid flow, and
when the prosthetic valve is in the closed state, the first and second coverings are configured to be positioned over respective portions of the first end of the body member to close off the first end fluid flow.

15. The prosthetic valve of claim 13, wherein, when the prosthetic valve is in the expanded configuration, the first and second elongate members are positioned across respective portions of the first end of the body member.

16. The prosthetic valve of claim 13, wherein, when the prosthetic valve is in the expanded configuration, the first and second elongate members comprise a stacked configuration and are positioned across a same portion of the first end of the body member.

17. The prosthetic valve of claim 16, wherein:
the first end and second end of the first elongate member are coupled to opposing positions on the first end of the body member, and
the first end and the second end of the second elongate member are coupled to opposing positions on the first end of the body member.

18. The prosthetic valve of claim 17, wherein, when the prosthetic valve is in the expanded configuration, the body member forms a cylindrical shape, and the first and second elongate members are configured to be positioned across a diameter of the body member.

19. The prosthetic valve of claim 13, wherein:
the first covering comprises a first leaflet and the second covering comprises a second leaflet, the first leaflet being coupled to the first elongate member along a longitudinal dimension of the first elongate member and the second leaflet being coupled to the second elongate member along a longitudinal dimension of the second elongate member, and
when the prosthetic valve is in the expanded configuration and the closed state, the first and second leaflets are each configured to cover a corresponding portion of the first end of the body member to close off fluid flow through the first end.

20. The prosthetic valve of claim 13, wherein:
the first and second coverings are each rotatably coupled to the respective elongate member, and
when the prosthetic valve is in the open state, the first and second coverings are configured to be pivotally rotated around the respective elongate member and away from the first end of the body member.
